SEQUENCE LISTING (11 sequences)

SEQ ID NO: 1 - protein, 5 aa, Artificial Sequence
  Peptide
KKKKD

SEQ ID NO: 2 - protein, 6 aa, Artificial Sequence
  Peptide
VKRKKK

SEQ ID NO: 3 - protein, 9 aa, Artificial Sequence
  Peptide
KKARAAVDA

SEQ ID NO: 4 - protein, 8 aa, Artificial Sequence
  Peptide
KAASWVGR

SEQ ID NO: 5 - protein, 7 aa, Artificial Sequence
  Peptide
KKARCNH

SEQ ID NO: 6 - protein, 6 aa, Artificial Sequence
  Peptide
AAVCNH

SEQ ID NO: 7 - protein, 7 aa, Artificial Sequence
  Peptide
AVDACNH

SEQ ID NO: 8 - protein, 7 aa, Artificial Sequence
  Peptide
ASAVCNH

SEQ ID NO: 9 - protein, 7 aa, Artificial Sequence
  Peptide
AVWACNH

SEQ ID NO: 10 - protein, 6 aa, Artificial Sequence
  Peptide
KKKRKV

SEQ ID NO: 11 - protein, 4 aa, Artificial Sequence
  Peptide
CVYC

(19) United States
(12) Patent Application Publication
(10) Pub. No.: US 2007/0027075 A1
(43) Pub. Date: Feb. 1, 2007

(54) Compositions and methods for targeted drug delivery

(76) Inventor: David B. Smithrud, Cincinnati, OH (US)

Correspondence Address: FROST BROWN TODD, LLC, 2200 PNC CENTER, 201 E. FIFTH STREET, CINCINNATI, OH, 45202, US

(21) Appl. No.: 10/560,121

(22) PCT Filed: Jun. 9, 2004
(86) PCT No.: PCT/US04/18301

§ 371 Date: May 23, 2006

Related U.S. Application Data
(60) Provisional application No. 60/477,091, filed on Jun. 9, 2003.

(51) Int. Cl. A61K 38/17; A61K 31/724; A61K 48/00; A61K 31/58; A61K 31/395

(52) U.S. Cl. 514/12; 514/183; 514/58; 514/44; 514/176

(57) ABSTRACT

The present invention provides for methods and compositions for transporting agents and macromolecules across biological membranes. In one embodiment, the invention relates to a method for enhancing transport of a selected agent across a biological membrane, wherein a biological membrane is contacted with a composition containing a biologically active rotaxane capable of selectively transporting the selected agent. The host-rotaxane is effective to impart to the agent an amount transport and/or rate of trans-membrane transport across a biological membrane that is greater than the amount and/or rate of trans-membrane transport of the agent without the host-rotaxane.

This application claims the benefit of U.S. Provisional Patent Application Ser. No. 60/477,091, filed Jun. 9, 2003, which application is hereby incorporated by reference in its entirety.

### FIELD OF THE INVENTION

The present invention relates to synthetic host-rotaxanes, and in particular novel synthetic host-rotaxanes that engage in molecular recognition events with a guest molecule to yield a host-guest complex. The present invention also provides for methods and compositions for transporting agents and macromolecules across biological membranes. In one embodiment, the invention pertains to a method for enhancing transport of a selected agent across a biological membrane, wherein a biological membrane is contacted with a composition containing a biologically active rotaxane capable of selectively transporting the selected agent. These host-rotaxanes can further be used in purification, transport, and catalysis events.

### BACKGROUND OF THE INVENTION

Rotaxanes are molecules comprising a linear component with a bulky group at each terminal end, and a circular “wheel” component. The wheel component encircles and is retained around the linear component by virtue of bulky end groups at either end of the linear component. Herein, the circular component will be referred to as a wheel component, and the bulky end groups present at each end of the linear component will be referred to as blocking groups. The blocking groups should be of sufficient steric size to prevent the “de-threading,” or removal of the circular component from the linear component of the rotaxane. The wheel component encircling the linear component of the rotaxane can be free to slide along, and/or pirouette around the linear component of the host-rotaxane.

Until now, current interest and research into rotaxanes has been limited to manipulating the linear and wheel components of the rotaxane to encourage and create desired interactions between the wheel and the linear components of the rotaxane. For example, U.S. Pat. No. 5,538,655 to Fauteaux, et al., which is herein incorporated by reference, describes using the wheel component of a rotaxane to transport ions back and forth along the linear component of the rotaxane through an electrolyte composition within an electrolyte cell.

One current area of interest in contemporary chemistry research is the development and synthesis of synthetic hosts. Synthetic hosts, such as cyclophanes have demonstrated that rigid, preformed aromatic pockets can be used for binding a guest. However, using convergent functional groups in combination with, for example, a hydrophobic pocket, enhances recognition of a targeted binding constituent. Besides combining the necessary functional groups needed to form noncovalent interactions with a guest, a convergent arrangement can also activate the functional groups by, for example desolvation or electronic destabilization.

Although beneficial to guest binding, the construction of many synthetic hosts has failed to provide functional groups that are truly convergent in that they point towards the binding structure on the host molecule. Another problem with synthetic hosts is that the spatial arrangement of functional groups used for guest recognition is limited by the assembly of atoms through covalent bond formation, which defines the strict dimensions of the host, allowing for little or no flexibility.

Once an appropriate synthetic host design has been determined, its possible uses should be ascertained. One area of interest relates to using the synthetic hosts to provide protein-like function in biological environments, such as, cellular transport. Creating cellular transport agents, however, can be a challenge. Ideally, such an agent should bind a guest molecule strongly and be reasonably soluble in the varying environments found throughout a cell, as well as those areas surrounding the outside of the cell. The transport agent should also shield features of the guest molecule that may prevent membrane passage, such as an anionic charge.

Current transport delivery methods include covalently linking a desired molecule to a transporting peptide, generally a viral coat protein, polylysine, or polyarginine, which carries the guest across the cell membrane. A further delivery method includes using polyliposomes or polycationic groups, which utilize noncovalent interactions to surround the guest molecule to make it chemically susceptible to transport the guest across a cell membrane and into a cell. Although currently used, the above-listed methods of cell delivery are not optimal because the binding area between the guest and the transport agent are not specifically designed for the guest. Additionally, each of the above-listed delivery methods is potentially toxic to the cell and the means of transport, i.e., endocytosis, can degrade the guest.

Consequently, a significant need exists for a synthetic host that can provide flexible and convergent functional groups for improved guest recognition. A further need exists for a synthetic host that is designed to recognize a targeted guest, can act as a transport agent, and is generally non-toxic to cells.

The present invention addresses these and other problems by providing a synthetic host-rotaxane having convergent functional groups (recognition elements), which adjust to accommodate a guest molecule and provide noncovalent interactions independent of the environment surrounding the host. The synthetic host-rotaxane of the present disclosure further provides a transport agent with a pre-designed, controlled binding area that can be transported across a natural or synthetic cell membrane when a guest molecule is present. Further the transport across the cell membrane can be accomplished with no noticeable toxicity to the cell.

Furthermore, a major hurdle for drug development continues to be poor drug delivery. A drug needs to be concentrated at diseased cells to reduce the damage to healthy cells and may need to penetrate cellular membranes. Satisfying these requirements severely limits the number of potential drugs and increases the costs of drug development. Life depends on the controlled transport of molecules across biological membranes. Although the strict limitation of membrane-permeable molecules maintains cell-health, it severely limits pharmaceutical research and drug development. New techniques such as combinatorial chemistry and phage display, combined with rapid throughput screening, are ever increasing the number of potential drug candidates and cell-targeting agents. What remains a problem for many therapies is the poor cellular permeability of promising drugs and intracellular drug-stability, e.g., peptidic degradation or degradation of various drugs by the lysosome.

Breakthrough methods in the burgeoning field of cellular delivery agents have overcome some of the natural restrictions on permeability imposed by cellular membranes. Several of these promising transport systems are now in clinical trials. Artificial transporters can be conveniently divided into covalent and noncovalent approaches. Problems with the covalent attachment approach include the potential toxicity of the transfer-peptides and polycationic compounds and the covalent attachment may interfere with cellular activity. Furthermore, endocytosis may be involved, which can lead to drug degradation. Most noncovalent approaches involve encapsulation of a guest within natural or synthetic vectors. Transport appears to occur through endocytosis, which can lead to DNA degradation upon fusion with the lysosome. Other general problems with this noncovalent approach are that the synthetic vectors can be toxic (especially cationic vectors) and have to stay assembled prior to and during transport.

Vast time, effort, and resources have gone into developing drugs and identifying drug-targets. However, getting drugs to their targets is still a major hurdle in drug development and keeps these two promising research fields separated. Antibodies have the ability to selectively recognize the unique features found on the surfaces of cancerous cells. Several therapies exploit this feature to bring drugs or prodrugs to tumors. For example, traditional chemotherapeutic agents have been limited by their inability to target cancer cells over healthy cells. The Tumor-Activated Prodrug (TAP) therapy enhances selectivity by using prodrugs that are converted into active agents predominately in cancer cells through spontaneous chemical transformations or through a metabolic process, such as tumor-specific enzymic catalysis. The unpredictable expression levels of appropriate enzymes in cancer cells have stymied research into selective catalysis. The unique chemical conversion of a prodrug into a drug within cancer cells has shown more promise. Problems encountered with this approach include achieving the fine balance between prodrug and drug activity and cancer cell selectivity. The prodrug should not significantly attack healthy cells and only be converted to the drug inside the cancer cell. Furthermore, most prodrugs need to be cell membrane permeable, The released drug itself should also be cell membrane permeable because not all tumor cells are able to modify the prodrug. The released drug needs to enter and kill these cancer cells (the bystander effect), as well.

The Antibody Directed Enzyme Prodrug Therapy (ADEPT) is a powerful method for bringing drugs selectively to targeted cells, e.g., cancer cells. Cancer cells contain unique antigens on their surfaces, which can be selectively bound by antibodies. Antibodies (Ab) and their drug-conjugates are limited by poor uptake into tumor cell. The ADEPT method, however, separates cell recognition from drug delivery. Antibodies are covalently linked to enzymes that convert prodrugs to drugs. After the antibody-enzyme conjugate is administered and binds to cancer-cells, prodrugs are given, which become localized at cancer cells and converted to drugs. The ADEPT method is more complex than simple prodrugs, which naturally results in several additional problems. One of the more severe problems is the potential immunogenicity of the antibody and enzyme. Fortunately, the antibody can be ‘humanized’ to lower their immunogenicity. Other problems with the ADEPT method include the enzyme should not be active prior to tumor recognition (a clearance step, to remove the conjugate, is used before prodrug administration), the large size of the protein conjugate reduces its diffusion rate (especially problematic in larger tumors), and the conjugation can reduce the enzyme's catalytic activity.

One of the greatest limitations of cancer chemotherapy is the severe side effects accompanying the use of some of the most broadly active antitumor agents. For example, anthracycline anticancer compounds, such as doxorubicin, have a very wide spectrum of anticancer activity, but their side effects, when administered systemically, include significant myelosuppression, gastrointestinal toxicity with acute nausea and vomiting, local tissue necrosis that may require skin grafting in some cases, and dose-dependent cardiotoxicity often resulting in irreversible cardiomyopathy with serious congestive heart failure. A new drug delivery system for cytotoxic drugs that can target the drug specifically to tumor cells would not only eliminate these side effects but also increase the effectiveness of the drug against the tumor by preventing drug absorption by other tissues.

### BRIEF SUMMARY OF THE INVENTION

The present approach utilizes rotaxane architecture to obtain synthetic hosts, which have convergent functional groups (recognition elements) that can adjust to interact with a specific guest molecule or series of guest molecules. A synthetic host-rotaxane comprises a linear component that is disposed inside a wheel component to form a host-rotaxane. Blocking groups are present at a first and second terminal end of the linear component, wherein the blocking groups are of sufficient size to prevent the linear component of the host-rotaxane from de-threading from the wheel component. Further, at least one of the blocking groups on the first or second terminal end of the linear molecule of the host-rotaxane comprises a guest binding element for associating with a desired guest molecule to form a host-guest complex. The wheel component of the host-rotaxane may further comprise at least one covalently attached recognition element. The attached recognition element(s) may further be in a convergent arrangement that points towards the guest binding element of the host-rotaxane.

The present disclosure also includes a method of conducting a molecular recognition event comprising the steps of (a) providing a host-rotaxane solution where the host-rotaxane solution contains at least one host-rotaxane having a guest binding element on a terminal end of the host-rotaxane for associating a guest molecule; (b) introducing a guest molecule into the host-rotaxane solution; and (c) associating the host-rotaxane so that the guest molecule and host-rotaxane combine to form a host-guest complex. The molecular recognition event can further include the steps of transporting at least a portion of the host-guest complex across a cell membrane and releasing the guest molecule from the guest binding element into a cell.

The disclosure further includes a method of purifying a multi-constituent solution, comprising the steps of (a) providing a multi-constituent solution; (b) adding at least one host-rotaxane having a guest binding element constructed to target a specific constituent present in the multi-constituent solution; (c) associating at least one targeted constituent with the host-rotaxane to form a host-guest complex; (d) and separating the host-guest complex from the multi-constituent solution. The disclosure further provides a method of synthesizing the host-rotaxanes of the present disclosure.

Previous drugs that did not meet the cell-permeability requirement can be used and new drugs will no longer need a guiding mechanism or be modified beyond the addition of a fluorescein tag for cell-permeability. Having a “universal” delivery method would be significantly cheaper than developing a unique transporter for each drug,

The present invention also provides for a new approach to overcome some of these problems. The inventor's innovation is the creation of a host-rotaxane composition that brings low molecular compounds and small peptides into the cytoplasm and nucleus of eukaryotic cells through noncovalent complexes. This universal delivery method is not limited to cancers or diseases. The host-rotaxanes may become the key component of a universal therapy that connects a wide assortment of drugs with cellular targeting agents.

These compositions can also be used with antibodies or other cellular targeting agents, currently used in various therapies, to deliver a large variety of drugs selectively into target cells, such as cancer cells. The antibodies or other cellular targeting agent bring the host-rotaxane composition to the targeted cells through linkers. The linkers are engineered to break once the antibody or other cellular targeting agent associates with the targeted cells. The composition opens the target cell(s) or tumor to fluoresceinated drugs or prodrugs, and can deliver these materials deep within the solid tumor.

Accordingly, the present invention provides compositions for the effective delivery of therapeutic substances into the cytoplasm of targeted cells, as well as methods of producing the compositions, methods of delivery using the compositions, and methods of treating cancer.

The present invention provides for a method for delivery of an agent into a cell, the method comprising the steps of: i) providing a rotaxane composition specific for recognizing the agent, and ii) contacting the cell with the rotaxane under conditions so as to effect delivery of the agent into the cell.

The present invention provides for rotaxane compositions and methods effective to increase the rate at which a conjugated biologically active agent is transported through a biological membrane relative to the rate at which the biologically active agent can be transported through the biological membrane in unconjugated form. The present invention provides for rotaxane compositions and methods effective to increase the amount of conjugated biologically active agent that is transported through a biological membrane relative to the amount of biologically active agent that can be transported through the biological membrane in unconjugated form.

The target-binding moiety may be linked to the rotaxane by a linking moiety, which may impart conformational flexibility within the conjugate and facilitate interactions between the target-binding moiety and its biological target. In one embodiment, the linking moiety is a cleavable linker, e.g., containing a linker group that is cleavable by an enzyme or by solvent-mediated cleavage, such as an ester, amide, or disulfide group. In another embodiment, the cleavable linker contains a photocleavable group.

In another aspect, the invention includes a pharmaceutical composition for delivering a biologically active agent across a biological membrane. The composition comprises a biologically active agent and at least one transport rotaxane as described herein, and a pharmaceutically acceptable carrier. The rotaxane is effective to impart to the agent a rate of trans-membrane transport that is greater than the trans-membrane transport rate of the agent in non-conjugated form. In another aspect, the invention includes a therapeutic method for treating a mammalian subject, particularly a human subject, with a pharmaceutical composition as above.

The methods provided can be used for treating or preventing a disease, the method comprising administering to a subject in Which such treatment or prevention is desired the pharmaceutical composition described herein, in an amount sufficient to treat or prevent the disease in the subject. For example, the disease to be treated may include diabetes, cancer, respiratory ailments, neurodegenerative disorders, cardioplegia, and/or viral infections.

Further related inventions are the use of translocating rotaxanes in the following methods: a method to enhance the movement of an active agent across a lipid membrane; a method to enhance the uptake of an active agent into a cell; a method to enhance the uptake of an active agent across a cell layer; a method to enhance the uptake of an active agent into an epithelial cell; a method to enhance the movement of an active agent across a lipid membrane; a method to enhance the uptake of an active agent into a cell; and a method to enhance the uptake of an active agent across a cell layer.

Another aspect of the present invention is a method to provide a method for diagnosing a pathological disorder by administration of an amount of a translocating peptide-active agent complex, wherein the active agent is a diagnostic agent, such that the systemic concentration of the diagnostic agent is effective to diagnose the pathological disorder.

Another aspect of the present invention is a method to provide a method for preventing a pathological disorder by administration of a translocating rotaxane and active agent, wherein the active agent is a prophylactic agent, such that the systemic concentration of the prophylactic agent is effective to prevent the pathological disorder.

Another aspect of the present invention is a method for treating a pathological disorder by administration of a translocating rotaxane and active agent, wherein the active agent is a therapeutic agent, such that the systemic concentration of the therapeutic agent is effective to treat the pathological disorder.

Another aspect of the present invention is a method to provide a method for diagnosing a pathological disorder by administration of a translocating rotaxane and active agent, wherein the active agent contains a diagnostic agent, such that the systemic concentration of the diagnostic agent is effective to diagnose the pathological disorder.

Another aspect of the present invention is a method to provide a method for preventing a pathological disorder by administration of a translocating rotaxane and active agent, wherein the active agent contains a prophylactic agent, such that the systemic concentration of the prophylactic agent is effective to prevent the pathological disorder.

Another aspect of the present invention is a method to provide a method for treating a pathological disorder by administration of a translocating rotaxane and active agent, wherein the active agent contains a therapeutic agent such that the systemic concentration of the therapeutic agent is effective to treat the pathological disorder.

These and other objects and advantages of the present invention shall be made apparent from the accompanying drawings and the description thereof.

It must be noted that as used herein and in the appended claims, the singular forms “a,” “and,” and “the” include plural reference unless the context clearly dictates otherwise. Thus, for example, reference to “a gene” is a reference to one or more genes and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are incorporated herein by reference for the purpose of describing and disclosing, for example, the constructs and methodologies that are described in the publications which might be used in connection with, the presently described invention. The publications discussed throughout the text are provided solely for their disclosure prior to the filing date of the present application. Nothing herein is to be construed as an admission that the inventor is not entitled to antedate such disclosure by virtue of prior invention.

### BRIEF DESCRIPTION OF THE DRAWINGS

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1 provides examples of host-rotaxanes that can be constructed according to the present disclosure.

FIG. 2 depicts a method of constructing a DCC-rotaxane.

FIG. 3 illustrates a synthesis method for a wheel component of a host-rotaxane.

FIG. 4 illustrates synthesizing and threading a wheel component onto a linear component of a host-rotaxane, as well as methods of attaching recognition elements to a wheel component of a host-rotaxane.

FIG. 5 demonstrates a method of synthesizing a calixarene guest binding element and attaching it to a host-rotaxane.

FIG. 6 describes a method of synthesizing a non-cyclic aromatic guest binding element.

FIG. 7 illustrates a method for synthesizing a cyclic aromatic guest binding element.

FIG. 8 illustrates various synthesis methodologies for attaching a guest binding element to a host-rotaxane.

FIG. 9 depicts a schematic showing the steps involved with one embodiment of the Antibody Directed Cellular Transport method designed to deliver drugs or prodrugs selectively into cells.

FIG. 10 depicts a schematic showing the steps involved with another embodiment of the Antibody Directed Cellular Transport method designed to deliver drugs or prodrugs selectively into cells. Delivery of the toxin-rotaxane into a cell requires breaking the noncovalent rotaxane-Fl-antibody interaction (K_{Rotaxane·Fl-Ab})—

FIG. 11 depicts a schematic showing that toxins are linked to the rotaxane, and these rotaxanes would be selectively delivered to cancerous cells by Fl-antibodies. Delivery of the toxin-rotaxane into a cell requires breaking the rotaxane-Fl-antibody interaction K_{Rotaxane-Fl-Ab}).

FIG. 12 depicts a schematic showing one embodiment where a linker joins the antibody to the rotaxane wherein the bond is stable enough to form a conjugate but breaks after the antibody binds the surface of the tumor cell, preferably triggered by light or pH change. The transporter will be derivatized with Z (part of the linker) and will prefer the tumor over serum. Preferably, it is nontoxic or of low toxicity once the tumor cells are killed or impaired.

FIG. 13 shows the structure of a linker. A variety of linkers can be constructed to fine-tune the hydrolysis rate. Changing linking orientation (o, m, or p) and the electronic property of the aromatic ring (X=C, N, or O) adjusts the hydrolysis rate at pH 7.5 and 6.0.

FIG. 14 shows a flow diagram of photocleavable linkers that contain a photosensitizer (λmax>600 nm, skin penetration window) and a covalent bond, which cleaves upon contact with the produced singlet oxygen. A. The first linker will have thiazolium and an enamine (Ab is antibody). B. Many other photosensitizers are available. C. Other cleavable alkenes, which are less susceptible to hydrolysis, are available.

FIG. 15 shows a schematic of where cell-transportation occurs when transporter and fluorescein are added separately to a buffered solution in a well containing cells on a slide.

FIG. 16 shows a schematic of peptide-rotaxanes that may be transporters with cell-selectivity by using peptides that target tumors. (A) Peptides can be attached to two possible amines. The most likely site is the amine available on the blocking group. This blocking group is available by using DCC-rotaxane 6. Shown attached is the nuclear localization sequence VKRKKKP. (B) Shown in the hatched boxes are the interactions that allow the delivery of a fluoresceinated compound and the covering of impermeable functional groups of the attached peptide, which allows the rotaxane to traverse the membrane.

FIG. 17 shows a schematic depicting tumor cells embedded in Matrigel used to determine the propensity the transporter has for tumors versus buffer. After a set time period, the tumor is sectioned and analyzed for fluorescence by scanning a tumor slice and by removing cores, extracting, and then analyzing the supernatant for fluorescence.

FIG. 18 is a plot showing that rotaxane 3 binds Fl-Ab (anti-goat IgG) in (A) water (K_{A}=8×10⁵ M⁻¹, phosphate, pH 7) and (B) fetal bovine serum (K_{A}=1×10⁴ M⁻¹). Both aspects of the ADCT method have been demonstrated: (i) cellular transport and (ii) rotaxane.Fl-Ab complexation.

FIG. 19 depicts HPLC traces of transporter 2 exposed to fetal bovine serum (95%/5% DMSO at room temperature). Transporter was recovered via extraction. After 6 days, only a small percentage of the transporter (<15%) decomposed, the products of which are indicated by the open arrows.

FIG. 20 is a diagrammatic depiction of testing the ADCT method on tumors grown in Matrigel. After chemical activation (lowering of the solution's pH or light activation), the procedures are used to determine successful Fl-drug or Fl-prodrug delivery.

FIG. 21 depicts a flow diagram of one method of using the rotaxanes in treatment of cancer using a fluorescein labeled antibody directed towards the cancer cells and the treating the labeled cells with a rotaxane and then with a drug labeled with a marker is transported into the cell by the rotaxane transporter. The procedures are used to determine successful Fl-drug or Fl-prodrug delivery.

FIG. 22 shows the flow diagram of a rotaxane synthetic scheme.

FIG. 23 shows the flow diagram of a rotaxane synthetic scheme.

FIG. 24 shows the flow diagram of a rotaxane synthetic scheme.

FIG. 25 shows the flow diagram of a rotaxane synthetic scheme.

FIG. 26 shows the flow diagram of a rotaxane synthetic scheme.

FIG. 27 shows the flow diagram of a rotaxane synthetic scheme.

FIG. 28 shows the flow diagram of a rotaxane synthetic scheme.

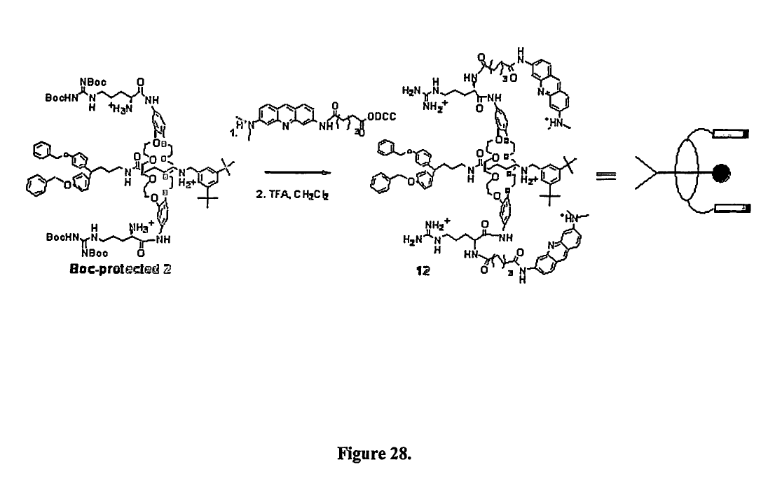

FIG. 29 shows the structure of (a) rotaxane 3; (b) rotaxane 2; (c) PKC inhibitor; (d) a model rotaxane; (e) DCC rotaxane with linker site; and (f) rotaxane 1.

### DETAILED DESCRIPTION OF THE INVENTION

It is to be understood that this invention is not limited to the particular methodology, protocols, constructs, formulae and reagents described and as such may vary. It is also to be understood that the terminology used herein is for the purpose of describing particular embodiments only, and is not intended to limit the scope of the present.

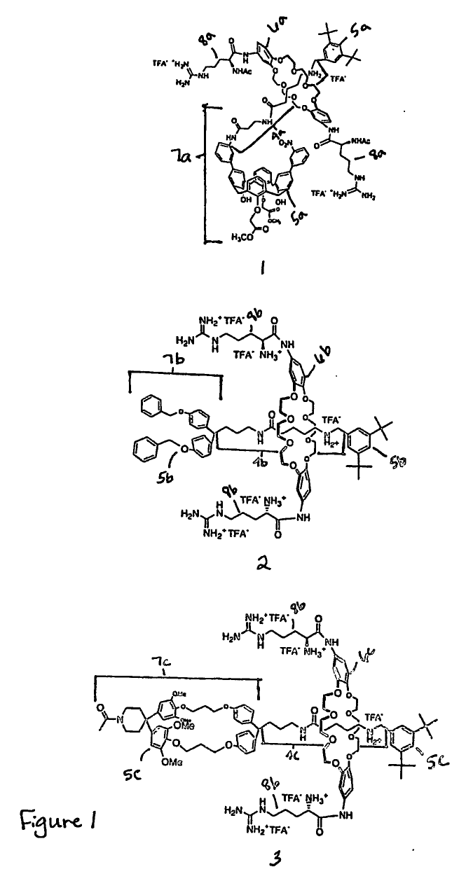

FIG. 1 depicts various host-rotaxanes of the present invention. Possible host-rotaxanes include, but are not limited to, calixarene-rotaxane 1, cleft-[2]rotaxane 2, and cyclophane-[2]rotaxane 3. The host-rotaxane of the present disclosure comprises two general components; a linear component 4a, 4b, and 4c connected to a blocking group 5a, 5b, and 5c at each terminal end and, a wheel component 6a, 6b, and 6c that encircles the linear component 4a, 4b, and 4c. The blocking groups 5a, 5b, and 5c should be of sufficient size to prevent the linear component 4a, 4b, and 4c from de-threading from the wheel component 6a, 6b, and 6c. The present disclosure generally contemplates the manipulation of at least one blocking group 5a, 5b, and 5c on the host-rotaxane structure to construct a guest binding element 7a, 7b, and 7c that is capable of binding a desired guest molecule. Additionally, the wheel component 6a, 6b, and 6c of the host-rotaxane may further comprise at least one recognition element 8a, 8b, and 8c, that is preferably in a convergent arrangement in relation to the guest binding element 7a, 7b, and 7c on the host-rotaxane.

As used herein, the term “rotaxane” refers to a macromolecular structure having a linear molecule (molecular axle) threaded through a macrocycle (molecular wheel). This structure is analogous to a ring positioned around a bone (or dumbbell), where movement of the ring over the bone (or dumbbell) occurs freely, but the ring cannot be easily removed from the ends of the bone (or dumbbell). As used herein, the phrase “linear molecule” refers to any molecule that can be inserted into a macrocycle. As used herein, the phrase “macrocycle” refers to a circular molecule with a diameter of a suitable size to allow for insertion of a linear molecule, such as, for example, rotaxanes, catenanes, carcerands, hemicarcerands, resorcinarenes, and calixarene capsules.

Macrocycles contemplated for use in the practice of the present invention comprise subunits linked in a cyclic manner. Subunits contemplated for use in the practice of the present invention include optionally substituted alkyl, cycloalkyl, oxyalkyl, aryl, heteroaryl, heterocyclic. In a preferred aspect, the macro cycle comprises optionally substituted aryl or heteroaryl subunits. The monomers are linked in a cyclic manner either directly or via substituents that are optionally attached to the subunits. Substituents contemplated for use in the practice of the present invention include alkyl, amide, carboxyl, hydroxy, hydroxyalkyl, oxyalkyl, amino, alkylamino. In another aspect, the macrocycle comprises optionally substituted oxyalkyl moieties, such as, for example, a crown ether.

A “molecular recognition event” occurs when a host-rotaxane and a guest molecule are introduced to one another and associate to form a host-guest complex.

A “host-guest complex” is a molecular entity comprising the host-rotaxane and its associated guest molecule.

A “guest molecule” (guest) is a chemical compound that is targeted by, and/or associates with a host-rotaxane during a molecular recognition event. Preferably, the guest is an active agent.

“Chemical entity”, as used herein, refers to cyclophanes, crown ethers, cryptands, resocirarenes, scaffolds, wheel components, guest molecules, as well as other compounds that are involved in molecular recognition events.

A “guest binding element” is a chemical entity attached as a blocking group on the linear component of the host-rotaxane of the present invention that may participate in the noncovalent binding of the guest molecule.

A “functional group” is a group of atoms attached to a chemical entity, which provides certain properties to that chemical entity (i.e., charge or reaction potential), as well as the reactions in which the chemical entity takes part. Any chemical entity disclosed herein as part of the host-rotaxane or guest molecule can have attached functional groups. The functional groups can act to facilitate association between the host-rotaxane and a guest molecule, and can be attached any portion of a host-rotaxane or a guest molecule. The addition or modification of functional groups to a chemical entity is known as “functionalization” of that particular chemical entity. Examples of functional groups that can be attached to the chemical entities of the present host-rotaxane and a guest molecule are aromatic rings, aliphatic moieties, carboxylates, ammonium ions, guanidinium ions, imidazolium ions, alcohols, amides, hydroxyls, phosphates, amines, carboxylic acids, anhydrides, and salts thereof, ketones, esters, olefins, as well as any others known in the art.

“Recognition elements” are functional groups attached to the wheel component of the host-rotaxane that interact and provide association between a host-rotaxane and guest molecule involved in a molecular recognition event. The interaction can occur between the recognition elements and a guest molecule, as well as between recognition elements and other chemical entities on the host-rotaxane, such as a guest binding element.

As used herein, a “derivatized construction” occurs when various functional groups are attached to a chemical entity. For example, a cyclophane consists of aromatic spacers and aliphatic linkers. Attaching carboxylates, ammonium ions, or other groups known in the art to the cyclophane forms derivatized constructions of cyclophane.

The term “active agent” is meant to refer to compounds that are therapeutic agents or imaging agents.

The term “therapeutic agent” is meant to refer to any agent having a therapeutic effect, including but not limited to chemotherapeutics, toxins, radiotherapeutics, or radiosensitizing agents.

The term “chemotherapeutic” is meant to refer to compounds that, when contacted with and/or incorporated into a cell, produce an effect on the cell, including causing the death of the cell, inhibiting cell division or inducing differentiation.

The term “toxin” is meant to refer to compounds that, when contacted with and/or incorporated into a cell, produce the death of the cell.

The term “radiotherapeutic” is meant to refer to radionuclides which when contacted with and/or incorporated into a cell, produce the death of the cell.

The term “radiosensitizing agent” is meant to refer to agents which increase the susceptibility of cells to the damaging effects of ionizing radiation or which become more toxic to a cell after exposure of the cell to ionizing radiation. A radiosensitizing agent permits lower doses of radiation to be administered and still provide a therapeutically effective dose.

The term “imaging agent” is meant to refer to compounds that can be detected.

The term “neoplasm” is meant to refer to an abnormal mass of tissue or cells. The growth of these tissues or cells exceeds and is uncoordinated with that of the normal tissues or cells and persists in the same excessive manner after cessation of the stimuli that evoked the change. These neoplastic tissues or cells show a lack of structural organization and coordination relative to normal tissues or cells that usually result in a mass of tissues or cells that can be either benign or malignant. Representative neoplasms thus include all forms of cancer, benign intracranial neoplasms, and aberrant blood vessels such as arteriovenous malformations (AVM), angiomas, macular degeneration, and other such vascular anomalies. As would be apparent to one of ordinary skill in the art, the term “tumor” typically refers to a larger neoplastic mass.

As used herein, neoplasm includes any neoplasm, including particularly all forms of cancer. This includes, but is not limited to, melanoma, adenocarcinoma, malignant glioma, prostatic carcinoma, kidney carcinoma, bladder carcinoma, pancreatic carcinoma, thyroid carcinoma, lung carcinoma, colon carcinoma, rectal carcinoma, brain carcinoma, liver carcinoma, breast carcinoma, ovary carcinoma, and the like. This also includes, but is not limited to, solid tumors, solid tumor metastases, angiofibromas, retrolental fibroplasia, hemangiomas, Karposi's sarcoma and the like cancers which require neovascularization to support tumor growth.

The phrase “treating a neoplasm” includes, but is not limited to, halting the growth of the neoplasm, killing the neoplasm, reducing the size of the neoplasm, or obliterating a neoplasm comprising a vascular anomaly. Halting the growth of the neoplasm refers to halting any increase in the size of the neoplasm or the neoplastic cells, or halting the division of the neoplasm or the neoplastic cells. Reducing the size of the neoplasm relates to reducing the size of the neoplasm or the neoplastic cells.

The term “subject” as used herein refers to any target of the treatment. Also provided by the present invention is a method of treating neoplastic cells that were grown in tissue culture. Also provided by the present invention is a method of treating neoplastic cells in situ, or in their normal position or location, for example, neoplastic cells of breast or prostate tumors. These in situ neoplasms can be located within or on a wide variety of hosts; for example, human hosts, canine hosts, feline hosts, equine hosts, bovine hosts, porcine hosts, and the like. Any host in which is found a neoplasm or neoplastic cells can be treated and is accordance with the present invention.

The term “subject” as used herein refers to any invertebrate or vertebrate species. The methods of the present invention are particularly useful in the treatment and diagnosis of warm-blooded vertebrates. Thus, the invention concerns mammals and birds. More particularly, provided is the treatment and/or diagnosis of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, e.g., poultry, such as turkeys, chickens, ducks, geese, guinea fowl, and the like, as they are also of economical importance to humans. Thus, provided is the treatment of livestock, including, but not limited to domesticated swine (pigs and hogs), ruminants, horses, poultry, and the like.

The terms “pharmaceutically acceptable”, “physiologically tolerable” and grammatical variations thereof, as they refer to compositions, carriers, diluents and reagents, are used interchangeably and represent that the materials are capable of administration to or upon a vertebrate subject without the production of undesirable physiological effects such as nausea, dizziness, gastric upset and the like.

The terms “bind”, “binding”, “binding activity” and “binding affinity” are believed to have well-understood meanings in the art. To facilitate explanation of the present invention, the terms “bind” and “binding” are meant to refer to protein-protein interactions that are recognized to play a role in many biological processes, such as the binding between an antibody and an antigen. Exemplary protein-protein interactions include, but are not limited to, covalent interactions between side chains, such as disulfide bridges between cysteine residues; hydrophobic interactions between side chains; and hydrogen bonding between side chains.

The terms “binding activity” and “binding affinity” are also meant to refer to the tendency of one protein or polypeptide to bind or not to bind to another protein or polypeptide. The energetics of protein-protein interactions are significant in “binding activity” and “binding affinity” because they define the necessary concentrations of interacting partners, the rates at which these partners are capable of associating, and the relative concentrations of bound and free proteins in a solution. The binding of a ligand to a target molecule can be considered specific if the binding affinity is about 1×10⁴ M⁻¹ to about 1×10⁶ M⁻¹ or greater.

The phrase “specifically (or selectively) binds”, for example when referring to the binding capacity of an antibody, also refers to a binding reaction which is determinative of the presence of the antigen in a heterogeneous population of proteins and other biological materials. The phrase “specifically (or selectively) binds” also refers to selective targeting of a targeting molecule.

The term “extracellular” as it relates to cleavage of the rotaxane molecule of the present invention refers to cleavage of the rotaxane molecule outside of a cell of the treated subject, such as, for example, in the gastrointestinal tract, in blood, in lymphatic fluid, peritoneal fluid, interstitial fluid, spinal fluid, synovial fluid, vaginal fluid or lung fluid and such similar space. The term “intracellular” as it relates to cleavage of the rotaxane molecule of the present invention refers to cleavage of the rotaxane molecule inside a cell in a treated subject.

The term “molecule” include any compound or salts thereof, whether naturally occurring or synthetically made, and includes a peptide, an oligopeptide, a polypeptide, a protein including a glycoprotein, a nucleic acid, whether DNA or RNA, a carbohydrate, a natural product such as a plant product, other polymers including synthetic polymers and fragments, a hormone, a chemical compound such as taxol, its analog or derivative, combinations and analogs thereof.

The term “operably linked” as used in reference to the linkage between the target-binding moieties and the cleavage site in the rotaxane molecule means that target-binding moieties are linked in such manner that, for example, upon cleavage of the rotaxane molecule at the cleavage site, the rotaxanes are capable of exhibiting one or more of its biological activities within the cellular membrane of the target cell.

The term “pharmaceutically acceptable carrier” as used herein means a carrier that is appropriate for the mode of delivery of the rotaxane molecule or composition containing the rotaxane molecule. For example, for parenteral administration, an acceptable carrier can be saline; for oral administration, an acceptable carrier may be a food product that is genetically engineered to contain the rotaxane molecule such as rice, milk, vegetables and the like, where the food product may have been processed or extracted. A pharmaceutically acceptable carrier is generally a non-toxic solid, semisolid or liquid filler, diluent, encapsulating material or formulation auxiliary of any conventional type. It is non-toxic to recipients at the dosages and concentrations employed and is compatible with other ingredients of the formulation. For example, the carrier for a formulation containing polypeptides preferably does not contain oxidizing agents and other compounds that are known to be deleterious to the half-life or shelf-live of the polypeptides. Suitable carriers include, but are not limited to: water, dextrose, glycerol, saline, ethanol, and combinations thereof. The carrier may contain additional agents such as wetting or emulsifying agents, pH buffering agents, or adjuvants, which enhance the effectiveness of the formulation. Other materials such as anti-oxidants, humectants, viscosity stabilizers, and similar agents may be added as necessary. Percutaneous penetration enhancers such as Azone may also be included. Compositions for oral administration herein may form solutions, suspensions, tablets, pills, capsules, sustained release formulations or powders.

The term “pharmaceutically acceptable salts” suitable for use herein include the acid addition salts (formed with the free amino groups of the polypeptide) and those that are formed with inorganic acids such as, for example, hydrochloric or phosphoric acids, or such organic acids as acetic, mandelic, oxalic, and tartaric. Salts formed with the free carboxyl groups may also be derived from inorganic bases such as, for example, sodium, potassium, ammonium, calcium, or ferric hydroxides, and such organic bases as isopropylamine, trimethylamine, and the like.

The term “treated subject” refers to the subject to which delivery of the rotaxane molecule of the present invention is intended so as to produce a biological effect including a diagnostic, prophylactic, therapeutic or nutritional effect. Such treated subjects include, but is not limited to: humans, non-human animals such as farm animals including cattle, pigs, goats and horses, and domestic animals such as dogs and cats; as well as rodents; non-human primates; birds such as chickens; plants; microorganisms; parasites; and fish. A “treated subject” may include two subjects as, for example, where a rotaxane molecule containing a cleavage site specific to a microorganism (hereafter, a “targeted microorganism”) is administered to a subject and the microorganism transits through in the GI tract of the subject. The rotaxane molecule may be cleaved intracellularly by the targeted microorganism or released intact by the targeted microorganism for cleavage by the “treated subject” enzyme, that is, an enzyme of the subject. For example, if the rotaxane molecule carries a detectable signal, such as green fluorescent protein, for example, that is activated upon cleavage, presence of the green fluorescent protein will indicate presence of the microorganism in the gut of a human. The terms “individual,” “subject,” “patient,” and “treated subject” are used interchangeably herein.

The “target-binding moiety” or “targeting agent” may include an immunoglobulin, an integrin, an antigen, a growth factor, a cell cycle protein, a cytokine, a hormone, a neurotransmitter, a receptor or fusion protein thereof, a blood protein, an antimicrobial, or any fragment, or structural or functional analog thereof. In addition, the target itself may be an immunoglobulin, an integrin, an antigen, a growth factor, a cell cycle protein, a cytokine, a hormone, a neurotransmitter, a receptor or fusion protein thereof, a blood protein, an antimicrobial, or any fragment, or structural or functional analog thereof.

For example, in one embodiment of the invention, the target-binding moieties may be derived from human or non-human polyclonal or monoclonal antibodies. Specifically, these antibodies (immunoglobulins) may be isolated, recombinant and/or synthetic human, primate, rodent, mammalian, chimeric, humanized or CDR-grafted, antibodies and anti-idiotype antibodies thereto. Such moieties can be produced by enzymatic cleavage, synthetic or recombinant techniques, as known in the art and/or as described herein. Additionally, these binding moieties can also be produced in a variety of truncated forms in which various portions of antibodies are joined together chemically by conventional techniques, or prepared as a contiguous protein using genetic engineering techniques. As used presently, an “antibody,” “antibody fragment,” “antibody variant,” “Fab,” and the like, include any protein- or peptide-containing molecule that comprises at least a portion of an immunoglobulin molecule, such as but not limited to at least one CDR of a heavy or light chain or a ligand binding portion thereof, a heavy chain or light chain variable region, a heavy chain or light chain constant region, a framework region, or any portion thereof, or at least one portion of a receptor or binding protein, which can be incorporated into a pseudo-antibody of the present invention. Such antibody optionally further affects a specific ligand, such as but not limited to, where such antibody modulates, decreases, increases, antagonizes, agonizes, mitigates, alleviates, blocks, inhibits, abrogates and/or interferes with at least one target activity or binding, or with receptor activity or binding, in vitro, in situ and/or in vivo. In one embodiment of the invention, such antibodies, or functional equivalents thereof, may be “human,” such that they are substantially non-immunogenic in humans. These antibodies may be prepared through any of the methodologies described herein, including the use of transgenic animals, genetically engineered to express human antibody genes. For example, immunized transgenic mice (xenomice) that express either fully human antibodies, or human variable regions have been described. WO 96/34096, published Oct. 31, 1996. In the case of xenomice, the antibodies produced include fully human antibodies and can be obtained from the animal directly (e.g., from serum), or from immortalized B-cells derived from the animal, or from the genes encoding the immunoglobulins with human variable regions can be recovered and expressed to obtain the antibodies directly or modified to obtain analogs of antibodies such as, for example, Fab or single chain Fv molecules.

The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. The present invention thus encompasses antibody fragments capable of binding to a biological molecule (such as an antigen or receptor) or portions thereof, including but not limited to Fab (e.g., by papain digestion), Fab′ (e.g., by pepsin digestion and partial reduction) and F(ab′)₂ (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments. See, e.g., CURRENT PROTOCOLS IN IMMUNOLOGY, (Colligan et al., eds., John Wiley & Sons, Inc., NY, 1994-2001).

As with antibodies, other peptide moieties that bind a particular target protein or other biological molecule (target-binding peptides) are encompassed by the pseudo-antibody disclosed herein. Such target-binding peptides may be isolated from tissues and purified to homogeneity, or isolated from cells which contain the target-binding protein, and purified to homogeneity. Once isolated and purified, such target-binding peptides may be sequenced by well-known methods. From these amino acid sequences, DNA probes may be produced and used to obtain mRNA, from which cDNA can be made and cloned by known methods. Other well-known methods for producing cDNA are known in the art and may effectively be used. In general, any target-binding peptide can be isolated from any cell or tissue expressing such proteins using a cDNA probe such as the probe described above, isolating mRNA and transcribing the mRNA into cDNA. Thereafter, the protein can be produced by inserting the cDNA into an expression vector, such as a virus, plasmid, cosmid, or other vector, inserting the expression vector into a cell, proliferating the resulting cells, and isolating the expressed target-binding protein from the medium or from cell extract as described above. Alternatively, target-binding peptides may be chemically synthesized using the sequence described above and an amino acid synthesizer, or manual synthesis using chemical conditions well known to form peptide bonds between selected amino acids. Analogues and fragments of target-binding proteins may be produced by chemically modification or by genetic engineering. These fragments and analogues may then be tested for target-binding activity using known methods. See, e.g., U.S. Pat. No. 5,808,029 to Brockhaus et al., issued Sep. 15, 1998.

Alternatively, target-binding peptides, including antibodies, may be identified using various library screening techniques. For example, peptide library screening takes advantage of the fact that molecules of only “peptide” length (2 to 40 amino acids) can bind to the receptor protein of a given large protein ligand. Such peptides may mimic the bioactivity of the large protein ligand (“peptide agonists”) or, through competitive binding, inhibit the bioactivity of the large protein ligand (“peptide antagonists”). Phage display peptide libraries have emerged as a powerful method in identifying such peptide agonists and antagonists. In such libraries, random peptide sequences are displayed by fusion with coat proteins of filamentous phage. Typically, the displayed peptides are affinity-eluted against an immobilized extracellular domain of an antigen or receptor. Successive rounds of affinity purification and repropagation may enrich the retained phages. The best binding peptides may be sequenced to identify key residues within one or more structurally related families of peptides. The peptide sequences may also suggest which residues may be safely replaced by alanine scanning or by mutagenesis at the DNA level. Mutagenesis libraries may be created and screened to further optimize the sequence of the best binders. See, e.g., WO 0024782, published May 4, 2000, and the references cited therein; U.S. Pat. No. 6,090,382 to Salfeld et al., issued Jul. 18, 2000; WO 93/06213, to Hoogenboom et al., published Apr. 1, 1993.

Other display library screening methods are known as well. For example, E. coli displays employ a peptide library fused to either the carboxyl terminus of the lac-repressor or the peptidoglycan-associated lipoprotein, and expressed in E. coli. Ribosome display involves halting the translation of random RNAs prior to ribosome release, resulting in a library of polypeptides with their associated RNAs still attached. RNA-peptide screening employs chemical linkage of peptides to RNA. Additionally, chemically derived peptide libraries have been developed in which peptides are immobilized on stable, non-biological materials, such as polyethylene rods or solvent-permeable resins. Another chemically derived peptide library uses photolithography to scan peptides immobilized on glass slides. These methods of chemical-peptide screening may be advantageous because they allow use of D-amino acids and other unnatural analogues, as well as non-peptide elements. See WO 0024782, published May 4, 2000, and the references cited therein.

Moreover, structural analysis of protein-protein interaction may also be used to suggest peptides that mimic the binding activity of large protein ligands. In such an analysis, the crystal structure may suggest the identity and relative orientation of critical residues of the large protein ligand, from which a peptide may be designed. These analytical methods may also be used to investigate the interaction between a receptor protein and peptides selected by phage display, which may suggest further modification of the peptides to increase binding affinity. Thus, conceptually, one may discover peptide mimetics of any protein using phage display and the other methods mentioned above. For example, these methods provide for epitope mapping, for identification of critical amino acids in protein-protein interactions, and as leads for the discovery of new therapeutic agents. See WO 0024782, published May 4, 2000, and the references cited therein.

Additionally, target-binding moieties produced synthetically are another alternative or additional moiety that may be included in the pseudo-antibody constructs of the present invention.

The nature and source of the target-binding moiety of the present invention is not limited. The following is a general discussion of the variety of proteins, peptides and biological molecules that may be used in the in accordance with the teachings herein. These descriptions do not serve to limit the scope of the invention, but rather illustrate the breadth of the invention. Thus, an embodiment of the present invention may target one or more growth factors, or, conversely, derive the target-binding moiety from one or more growth factors. Briefly, growth factors are hormones or cytokine proteins that bind to receptors on the cell surface, with the primary result of activating cellular proliferation and/or differentiation. Many growth factors are quite versatile, stimulating cellular division in numerous different cell types; while others are specific to a particular cell-type. The following presents several factors, but is not intended to be comprehensive or complete, yet introduces some of the more commonly known factors and their principal activities.

Preferably, the target-binding moiety is a protein selected from the group consisting of an antibody, a cytokine, a growth factor, a cell cycle protein, a blood protein, an integrin, a receptor, a neurotransmitter, an antigen, an anti-microbial agent, and any functional or structural equivalent of any of the foregoing. In another embodiment, the target-binding moiety is a protein that is a receptor or a functional portion of a receptor for a molecule selected from the group consisting of an antibody, a cytokine, a growth factor, a cell cycle protein, a blood protein, an integrin, a neurotransmitter, an antigen, an anti-microbial agent, and any functional or structural equivalent of any of the foregoing.

In addition to the growth factors discussed above, the present invention may target or use other cytokines. Secreted primarily from leukocytes, cytokines stimulate both the humoral and cellular immune responses, as well as the activation of phagocytic cells. Cytokines that are secreted from lymphocytes are termed lymphokines, whereas those secreted by monocytes or macrophages are termed monokines. Various cells of the body produce a large family of cytokines. Many of the lymphokines are also known as interleukins (ILs), because they are not only secreted by leukocytes, but are also able to affect the cellular responses of leukocytes. More specifically, interleukins are growth factors targeted to cells of hematopoietic origin.

The present invention may also incorporate or target a particular antigen. Antigens, in a broad sense, may include any molecule to which an antibody, or functional fragment thereof, binds. Such antigens may be pathogen derived, and be associated with either MHC class I or MHC class II reactions. These antigens may be proteinaceous or include carbohydrates, such as polysaccharides, glycoproteins, or lipids. Carbohydrate and lipid antigens are present on cell surfaces of all types of cells, including normal human blood cells and foreign, bacterial cell walls or viral membranes. Nucleic acids may also be antigenic when associated with proteins, and are hence included within the scope of antigens encompassed in the present invention. See SEARS, IMMUNOLOGY (W. H. Freeman & Co. and Sumanas, Inc., 1997).

For example, antigens may be derived from a pathogen, such as a virus, bacterium, mycoplasm, fungus, parasite, or from another foreign substance, such as a toxin. Such bacterial antigens may include or be derived from Bacillus anthracis, Bacillus tetani, Bordetella pertusis; Brucella spp., Corynebacterium diphtheriae, Clostridium botulinum, Clostridium perfringens, Coxiella burnetii, Francisella tularensis, Mycobacterium leprae, Mycobacterium tuberculosis, Salmonella typhimurium, Streptococcus pneumoniae, Escherichia coli, Haemophilus influenzae, Shigella spp., Staphylococcus aureus, Neisseria gonorrhoeae, Neisseria meningitidis, Treponema pallidum, Yersinia pestis, Vibrio choterae. Often, the oligosaccharide structures of the outer cell walls of these microbes afford superior protective immunity, but must be conjugated to an appropriate carrier for that effect.

Viruses and viral antigens that are within the scope of the current invention include, but are not limited to, HBeAg, Hepatitis B Core, Hepatitis B Surface Antigen, Cytomegalovirus B, HIV-1 gag, HIV-1 nef, HIV-1 env, HIV-1 gp41-1, HIV-1 p24, HIV-1 MN gp120, HIV-2 env, HIV-2 gp 36, HCV Core, HCV NS4, HCV NS3, HCV p22 nucleocapsid, HPV L1 capsid, HSV-1 gD, HSV-1 gG, HSV-2 gG, HSV-II, Influenza A (H1N1), Influenza A (H3N2), Influenza B, Parainfluenza Virus Type 1, Epstein Barr virus capsid antigen, Epstein Barr virus, Poxyiridae Variola major, Poxyizidae Variola minor, Rotavirus, Rubella virus, Respiratory Syncytial Virus, Surface Antigens of the Syphilis spirochete, Mumps Virus Antigen, Varicella zoster Virus Antigen and Filoviridae.

Other parasitic pathogens such as Chlamydia trachomatis, Plasmodium falciparum, and, Toxoplasma gonzdii may also provide antigens for, or be targeted by, the pseudo-antibody of the present invention. Numerous bacterial and viral, and other microbe-generated antigens are available from commercial suppliers such as Research Diagnostics, Inc. (Flanders, N.J.).

Toxins, toxoids, or antigenic portions of either, within the scope of the present invention include those produced by bacteria, such as diphteria toxin, tetanus toxin, botulin toxin and enterotoxin B; those produced by plants, such as Ricin toxin from the castor bean Ricinus cummunis. Mycotoxins, produced by fungi, that may serve in the present invention include diacetoxyscirpenol (DAS), Nivalenol, 4-Deoxynivalenol (DON), and T-2 Toxin. Other toxins and toxoids produced by or derived from other plants, snakes, fish, frogs, spiders, scorpions, blue-green algae, snails may also be incorporated in the pseudo-antibody constructs of the present invention.

Antigens included in the constructs of the present invention may also serve as markers for particular cell types, or as targets for an agent interacting with that cell type. Examples include Human Leukocyte Antigens (HLA markers), MHC Class I and Class II, the numerous CD markers useful for identifying T-cells and the physiological states thereof. Alternatively, antigens may serve as “markers” for a particular disease or condition, or as targets of a therapeutic agent. Examples include, Prostate Specific Antigen, Pregnancy specific beta 1 glycoprotein (SP1), Thyroid Microsomal Antigen, and Urine Protein 1. Antigens may include those defined as “self” implicated in autoimmune diseases. Haptens, low molecular weight compounds such as drugs or antibiotics that are too small to cause an immune response unless they are coupled with much larger entities, may serve as antigens when coupled to the compounds of the present invention.

The compositions of the present invention may also include an organic moiety to which, through the optional use of a linker, the target-binding moiety is attached. The organic moiety serves to position the target-binding moiety to optimize avidity, affinity, and/or circulating half-life. This moiety can be a hydrophilic polymeric group, a simple or complex carbohydrate, a fatty acid group, a fatty acid ester group, a lipid group, or a phospholipid group. More specifically, polyglycols are hydrophilic polymers that have one or more terminal hydroxy groups, such as polyethylene glycol, polypropylene glycol, polyvinyl pyrrolidone, homo-polyamino acids, hetero-polyamino acids, and polyamides. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

Particular aspects of the host-rotaxane will be discussed below.

Blocking Group Manipulation

In one aspect, the present disclosure is directed to the manipulation of at least one blocking group on either the first or second terminal end of the host-rotaxane to include a guest binding element. The guest binding element is attached to the linear component on the host-rotaxane as a blocking group and comprises a desired chemical entity that will associate with, and/or host a guest molecule. The guest binding element is essentially a pre-designed, controlled binding structure that complements a targeted guest.

The guest binding element, which is a blocking group attached to the linear component of the host-rotaxane, can be a cyclic or non-cyclic aromatic compound. Examples of suitable cyclic aromatic compounds are calixarenes, cyclophanes, cyclodextrins, resorcinarenes, as well as functionalized constructions thereof. Cyclic aromatic compounds as guest binding elements are most appropriately formulated for binding large and small aromatic or aliphatic groups in aqueous or non-aqueous polar solvents.

The cyclic aromatic compounds described above can host various compounds including, but not limited to proteins, peptides, amino acids, aromatic compounds, inorganic cations and anions, organic cations and anions, sugars, DNA, RNA, nucleotides, phosphates, phospholipids, fatty acids, steroids, isoprene derivatives, as well as other compounds known to be compatible with the aromatic compounds described above.

By way of example, calixarene used as a guest binding element can host various guest molecules including, N-Ac-L-Trp, indole, N-Ac-Gly, L-Trp, D-Trp, 1,5-DNS (1-[di-methylamino]-5-naphthalenesulfonate), fluorescein, and pyrene among other compounds known in the art. Cyclophane as a guest binding element can host various compounds such as, Trp, Ac-Trp, Ac-Trp-NH₂, indole, In(CH₂)₂CO₂H, Ac-Tip-Gly, Ac-Trp-Glu, Ac-Trp-Asp, Ac-Tip-Ala, Ac-Trp-Leu, Ac-Trp-Ile, 1,5-DNS, fluorescein, and pyrene, among others known in the art.

The guest binding element can further be a non-cyclic, aromatic moiety. Such moieties could, for example, be non-cyclic concave shaped moieties such as clefts, clips, or other scaffolds that contain functional groups known in the art such as peptidomimetic templates, as well as functionalized constructions thereof. Such guest binding elements can host a variety of guest molecules depending on the type of functional groups attached to the guest binding element.

Examples of guest molecules that can be hosted by a non-cyclic aromatic guest binding element include, but are not limited to proteins, peptides, amino acids, aromatic compounds, inorganic cations and anions, organic cations and anions, sugars, DNA, RNA, nucleotides, phosphates, phospholipids, fatty acids, steroids, isoprene derivatives, as well as other compounds known to be compatible with the non-cyclic hosts described above. By way of example, a cleft as a guest binding element can host a variety of compounds such as, Trp, Ac-Trp, Ac-Trp-NH₂, indole, In(CH₂)₂CO₂H (3-indolepropionic acid), Ac-Trp-Gly, Ac-Trp-Glu, Ac-Trp-Asp, Ac-Trp-Ala, Ac-Trp-Leu, Ac-Trp-Ile, 1,5-DNS, fluorescein, and pyrene as well as others known in the art.

The guest binding element can further be a cyclic or non-cyclic aliphatic ether. Cyclic aliphatic ethers that can be guest binding elements are, for example, crown ethers, podands, or other rings that have nitrogen or sulfur atoms, such as cyclic lactams, as well as functionalized versions thereof. Various non-cyclic aliphatic ethers, such as synthetic and naturally occurring ionophores, as well as polyether antibiotics can be guest binding elements. These cyclic and non-cyclic aliphatic ether moieties can host, for example, inorganic cations and anions, organic cations and anions, phosphates, phospholipids, polar compounds, as well as other compounds known in the art.

The guest binding element can further be a charged species. The guest binding element can be an anionic compound, such as carboxylates, phosphates, phosphonates, or sulfates, as well as functionalized constructions thereof. Additionally, the guest binding element can be a cationic compound, such as, an ammonium ion, guanidinium ion, or imidazolium ion, as well as functionalized constructions thereof.

A variety of functional groups can be attached to the guest molecules and guest binding elements, which can optimize association between the two chemical entities. Additionally, both a guest binding element and its targeted guest molecule can have attached functional groups, so long as those functional groups are compatible with one another. Selective guest molecule association can be obtained through coordinating functional groups present on the guest molecule and guest binding element, i.e., positive with negative charges, hydrogen-bonding donators with acceptors, aromatic surfaces with aromatic surfaces, as well as other combination recognized in the art. Functional groups that can be attached to guest binding elements are, for example, alcohols, amides, amines, ketones, esters, carboxylic acids, cationic groups (i.e., guanidinium ions and imidazoles), olefins, aromatic rings, and aliphatic moieties, as well as others known in the art.

Additionally, attaching recognition elements to the wheel of the host-rotaxane can optimize association between a guest molecule and a guest binding element. The recognition elements interact to promote association between the host-rotaxane and guest molecule, which can occur between the recognition elements and the guest molecule, as well as between the host-rotaxane and its attached recognition elements.

Interactions between a host-rotaxane and various guest molecules include all forms of noncovalent forces. Examples include, but are not limited to ionic bonds, hydrogen bonds, dipole-dipole interactions, and van der Waals forces.

The above-discussed guest binding elements, and any others known in the art, can successfully associate with a targeted guest molecule in various solvents and solvent systems of all polarities including, CHCl₃ (chloroform) DMSO (dimethylsulfoxide), DMF (dimethyl formamide), and H₂O, as well as combinations thereof. For example, the host-rotaxanes of the present disclosure can bind a variety of guest molecules in DMSO (100%) and H₂O (99%). The solvents can further be used in combination. Such combinations include, but are not limited to 80% DMSO and 20% H₂O, 50% DMSO and 50% H₂O, 25% DMSO and 75% H₂O, as well as 2% DMSO and 98% H₂O, as well as any others known in the art.

Wheel Manipulation

The host-rotaxanes of the present disclosure comprise a wheel component that can freely pirouette around and/or slide along the linear axis of the linear component of the host-rotaxane. In one aspect, the moveable wheel component of the host-rotaxane allows for adjustment of any present recognition element(s) in order to interact with a guest molecule. Such a construction allows the host-rotaxane and its associated guest molecule to maintain the strongest possible intermolecular interactions regardless of changes in the environment, i.e., a change in solvent conditions. For example, in non-polar environments, the wheel component can adjust to allow for contact between its polar recognition elements and the guest molecule associated with the host-rotaxane. Such contact promotes, for example, salt bridges, hydrogen bonds, or other noncovalent interactions to occur between, for example, the recognition element(s) and a guest molecule or recognition element(s) and a guest binding element. In aqueous environments, however, a different binding geometry generally occurs to allow for hydrophobic, and other interactions to occur between a guest binding element and a guest molecule.

This particular feature permits the host-rotaxanes of the present disclosure to strongly bind a desired guest molecule in multiple solvent systems, which makes the host-rotaxanes, as described herein, particularly well-designed for use as intercellular transport agents, which will be described below.

A further aspect of the present disclosure relates to manipulation of the wheel component of the host-rotaxane to include at least one recognition element. The recognition element can point towards the guest binding element, which activates it for guest molecule association. Attached recognition elements can interact favorably with a guest molecule alone or in concert with the guest binding element and/or wheel component present on the host-rotaxane.

The wheel component may contain no attached recognition elements, but preferably contains at least one recognition element. It is further preferred that the wheel component comprises at least two recognition elements. Additionally it is preferred that the recognition elements are oriented such that they point towards the guest binding element. This convergent arrangement facilitates the occurrence of noncovalent interactions between the recognition elements, and either the guest binding element or guest molecule.

Various recognition elements can be attached to the wheel component, depending on the targeted guest molecule and any desired noncovalent interactions. Suitable recognition elements are those that provide the desired interactions, such as, for example, carboxylates, ammonium ions, guanidinium ions, imidazolium ions, phosphates, alcohols, carboxylates, amides, sulfhydryls, aliphatic groups, aromatic compounds, as well as any other compounds known in the art. Possible noncovalent interactions between the recognition element/guest molecule or recognition element/guest binding element can be electrostatic interactions (salt bonds), hydrogen bonds, and dispersion interactions (London forces), as well as other noncovalent interactions known in the art.

These noncovalent interactions and host-rotaxanes can be tuned to enhance guest molecule association. Because the host-rotaxane structure and its attached recognition elements are flexible and the wheel component can slide and pirouette around the axle, the host-rotaxanes of the present invention can be programmed to bind, for example, a single guest or a class of guests. For example, host-rotaxanes with a long axle can bind aromatic carboxylic acids of different sizes and geometries, but shortening the axle can favor smaller aromatic carboxylic acids.

Further, fixing the wheel component at a specific distance from the guest binding element so that it cannot move along the linear component of the host-rotaxane can result in greater guest selectivity. The wheel component can be fixed, for example, by modifying the linear component, i.e., shortening the linear component or attaching functional groups to the linear component on either side of the wheel component. Additionally, the wheel component can be prevented from pirouetting around the linear component of the host-rotaxane by forming favorable intramolecular interactions between a recognition element of the wheel component and a guest binding element. Selectivity in guest molecule association can further be obtained through matching functional groups present on the guest molecule or guest binding element with recognition elements on the wheel component of the host-rotaxane, i.e., positive with negative charges, hydrogen-bond donators with acceptors, aromatic surfaces with aromatic surfaces, as well as other combination recognized in the art.

Host-rotaxanes may further comprise fluorophores or other marking compounds or materials known in the art to enable an observer to locate or view the presence of the host-rotaxane. These marking compounds may be coupled to the host-rotaxane structure by any method known in the art. Further, the guest molecule may comprise a fluorophore or other marking compound known in the art.

Molecular Recognition

The host-rotaxanes of the present invention engage in molecular recognition events. A molecular recognition event occurs when a host-rotaxane and a guest molecule are introduced to one another and associate to form a host-guest complex. In particular, a molecular recognition event occurs when a host-rotaxane with an attached suitable pre-designed guest binding element and any appropriate recognition elements are present, which can mimic an antibody, protein, or other binding structure known in the art, with sufficient specificity to target and associate with the targeted guest molecule.

A molecular recognition event can occur using any host-rotaxane construction described herein, including any of the previously described guest binding elements as well as any compatible recognition elements. These molecular recognition events can, for example, be used to engage in separation and/or purification events where a targeted molecule is separated from a solution containing multiple constituents, as host-rotaxanes have the ability, with great specificity, to associate with and separate targeted molecules from a multi constituent solution. A purification and/or separation event can then be completed by the host-rotaxane releasing the bound molecules using any method known in the art. For example, separation and/or purification events can be conducted to perform affinity chromatography, chiral resolution (enantiomeric enrichment), or any other separation and/or purification events known in the art. A host-rotaxane of the present disclosure that has undergone a molecular recognition event can further be used as a catalyst to increases the rate of a chemical reaction, without being consumed itself in the reaction

A molecular recognition event can further be used to perform protein-like functions, such as, for example, transport. In particular, host-rotaxanes can engage in cellular transport events across natural and/or synthetic membranes. The host-rotaxanes, as described herein, are successful as cellular transporters because, as previously discussed, they are configured to adopt favorable interactions depending on the operating environment of the host. In non-polar environments, like those found within a cell membrane, a host-rotaxane will adjust to allow any attached recognition elements to engage in noncovalent interactions such as salt bridges, hydrogen bonds, and other noncovalent interactions to maintain association of the guest molecule. In aqueous environments, however, like those found within a cell, a greater hydrophobic effect will occur and the guest binding element on the host-rotaxane will contract around the guest molecule using its hydrophobic moieties to bind the guest molecule within the guest binding element on the host-rotaxane. The host-rotaxane is further soluble in both the polar and non-polar environments found in a cell.

The above unique features combine to provide a host-rotaxane molecule that is not only soluble in the various environments found within a cell, but one that can also select and strongly bind a targeted guest molecule with a high degree of specificity. This allows a host-rotaxane to bind a targeted guest molecule and transport it across a cell membrane and into a cell to provide, for example, delivery of a desired compound into a cell. Additionally, the host-rotaxane uses noncovalent forces to carry a molecule into a cell, and will not interfere with the guest molecule's intercellular function.

The host-rotaxanes of the present invention can be used to transport any guest molecule so long as the guest molecule is sufficiently compatible to bind with the host-rotaxane and does not interfere with its transport, for example, across a cell membrane. The host-rotaxanes of the present disclosure can transport, for example, fluorescein or other fluorophores including fluorescein derivatized agents such as, for example, fluorescein-PKC inhibitor or a fluorescein tagged peptides, as well as others known in the art.

A molecular recognition and transport event, using the host-rotaxanes of the present disclosure, could further be used as a drug delivery agent that binds a targeted pharmaceutical or other therapeutic compound, such as, for example, a drug, an active peptide, or a protein-based drug, and transports it across a cell membrane. The host-rotaxane could further deliver the guest molecule to a desired location in a cell, such as, inside a nucleus, cytoplasm, or other cellular structure, i.e., a mitochondrion. By way of example, fluorescein-tagged compounds have been transported into a cell using the host-rotaxanes described herein. These tags can be released from guest molecules of the present invention using known release mechanisms, such as, for example, linkers containing disulfides or esters, among others known in the art.

The host-rotaxanes, as described herein, can effectively deliver agents through a plasma membrane into a cell cytoplasm, as well as through a nuclear envelope and into a nucleus. Additionally, the host-rotaxanes can further uniformly deliver an agent throughout an entire population of cells independent of cell differentiation. Further, functional groups on a guest binding element or a recognition element can be used as cellular targeting mechanisms for cell types or cellular compartments.

Release mechanisms can be employed to release a guest that is associated with a host-rotaxane. Such mechanisms can be any known in the art, such as those discussed above (i.e., disulfide or ester hydrolysis). Alternatively, the release could occur naturally by dilution or introducing the host-guest complex into a cell and using stronger or more desirable interactions provided by a biological compound to remove the guest molecule from the host-rotaxane. This aspect of the release can ensure that the transported compound is deposited into the cell at its intended location.

Synthesis of Host-rotaxanes

The host-rotaxanes of the present invention can be synthesized using any methods known in the art, such as those discussed below. The host-rotaxanes can further be synthesized using, for example, the combinatorial methods or dynamic combinatorial methods.

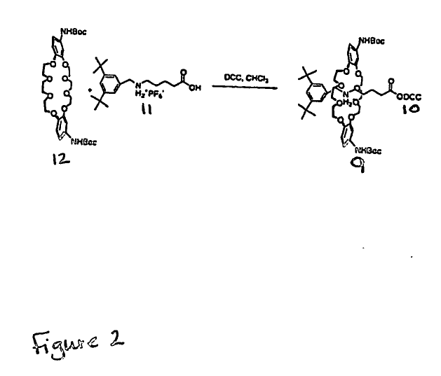

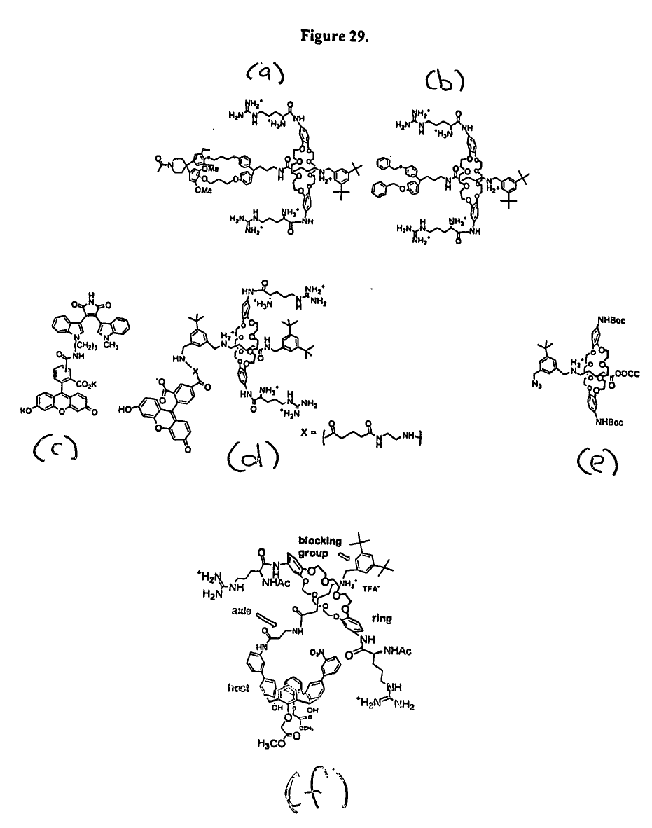

A convenient way to construct host-rotaxanes, as depicted by FIGS. 2 and 29 for example, is to first assemble DCC-rotaxane 9, which contains a DCC portion 10. As used herein, DCC refers to Dicyclohexylcarbodiimide. A desired blocking group can then be attached to the activated carbonyl of DCC-rotaxane 9, which contains a nucleophile known in the art, such as a primary amine, to react with DCC-activated acids. The blocking group can, for example, be a guest binding element, or any other blocking group known in the art. Further, recognition elements can be attached to the wheel component of the host-rotaxane either before or after attachment of the final blocking group by any method known in the art, including those discussed below.

Previous research presented in Facile Synthesis of Rotaxanes through Condensation Reactions of DCC-Rotaxanes Org. Lett. 2001, 16, 2485-2486, by Zehnder, D.; Smithrud, D. B., which is herein incorporated by reference, demonstrated that DCC-rotaxanes can be constructed by the addition of DCC to pseudorotaxanes composed of dibenzo-24-crown-8 (DB24C8) rings threaded onto an axle containing an ammonium ion and carboxylic acid. Referring now to FIGS. 2 and 29, DCC-rotaxane 9 can, for example, be synthesized in a similar manner except that a Boc(tert-butoxycarbonyl) protected diamino-DB24C8 ring is used. Adding, for example, axle component 11 and diamino-DB24C8 12f in CHCl₃, and then combining DCC can synthesize in particular, DCC-rotaxane 9.

The host-rotaxanes of the present invention can further contain more than one wheel component to form, for example, [3]rotaxanes, [4]rotaxanes, and [5]rotaxanes. Such construction can occur, for example, by using the DCC-rotaxane method with guest binding elements that have more than one nucleophile.

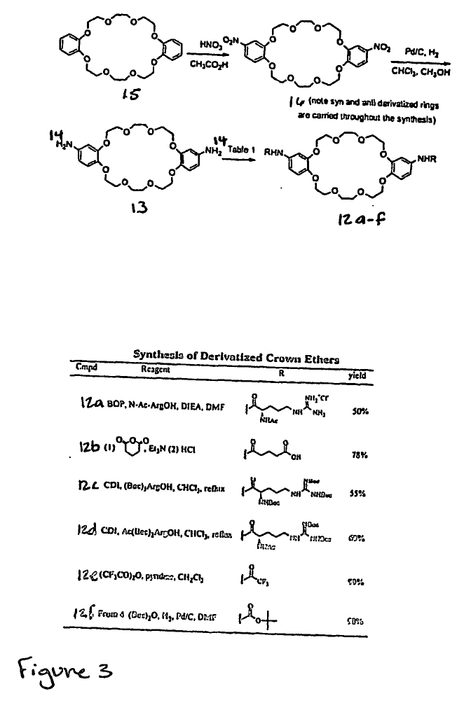

Referring now to FIG. 3, in order to synthesize the host-rotaxane with recognition elements on the host-rotaxane's wheel component, a di(aminobenzo)[24]crown-8 (diamino-DB24C8) 13 is formulated. This derivatized construction allows various desired recognition elements to be attached. As used herein, a derivatized DCC-rotaxane refers to a DCC-rotaxane having a group or groups attached that provide simplified attachment of desired recognition elements to a DCC-Rotaxane 9, i.e., the amino groups 14 located on the diamino-DB24C8 13. The diamino-DB24C8 13 synthetic route can begin with the nitration of DB24C8 15 with HNO₃ and CH₃CO₂H, which produces a mixture of syn and anti constitutional isomers of di(nitrobenzo)[24]crown-8 (dinitro-DB24C8) 16 (the syn isomer is shown in the figures for simplicity). The dinitro-DB24C8 16 can then be reduced to form diamino-DB24C8 13 in a CHCl₃ and methanol solution in the presence of 10-mol % Pd/C under H₂. Because diamino-DB24C8 13 is unstable, formation of Boc protected crown ether should be performed in situ with the reduction reaction.

Once the diamino-DB24C8 13 has been synthesized, recognition elements can be attached using any method known in the art. Examples of such recognition elements are shown in the below table.

TABLE I
Synthesis of Recognition Elements on Wheel

| Compound | Reagent | R | Yield |
|---|---|---|---|
| 12a | BOP, N-Ac-ArgOH, DIEA, DMF | 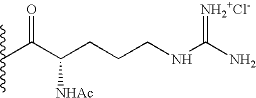 | 50% |
| 12b | 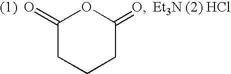 | 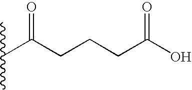 | 78% |
| 12c | CDI, (Boc)₃ArgOH, CHCl₃, reflux | 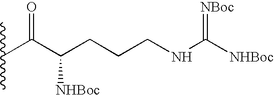 | 55% |
| 12d | CDI, Ac(Boc)₂ArgOH, CHCl₃, reflux | 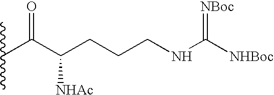 | 69% |
| 12e | (CF₃CO)₂O, pyridine, CH₂Cl₂ | 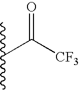 | 99% |
| 12f | (Boc)₂O, H₂, DMF | 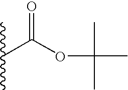 | 90% |

Crown ether 12a, di(N-acetylarginylaminobenzo)[24]crown-8, can be synthesized by using N-Ac-Arg-OH.HCl in DMF with BOP to facilitate the reaction. Crown ether 12b, di(4-carboxybutyrylaminobenzo)[24]crown-8, can be synthesized, for example, by adding glutaric anhydride to the diamino-DB24C8 13. A CDI-catalyzed coupling reaction with (Boc)₃-Arg-OH or AC-(Boc)₂-Arg-OH can yield di[(Boc)₃arginylaminobenzo][24]crown-8 (crown ether) 12c and di[N-acetyl(30c)₂arginylaminobenzo][24]crown-8 (crown ether) 12d, respectively. Di(trifluoroacetylaminobenzo)[24]crown-8 (crown ether) 12e can be obtained, for example, by reacting diamino-DB24C8 13 with trifluoroacetic anhydride in pyridine. Di(tert-butoxycarbonylaminobenzo)[24]crown-8 (crown ether) 12f can be obtained, for example, by reacting (Boc)₂O, H₂, Pd/C, and DMF with the dinitro-DB24C8 13.

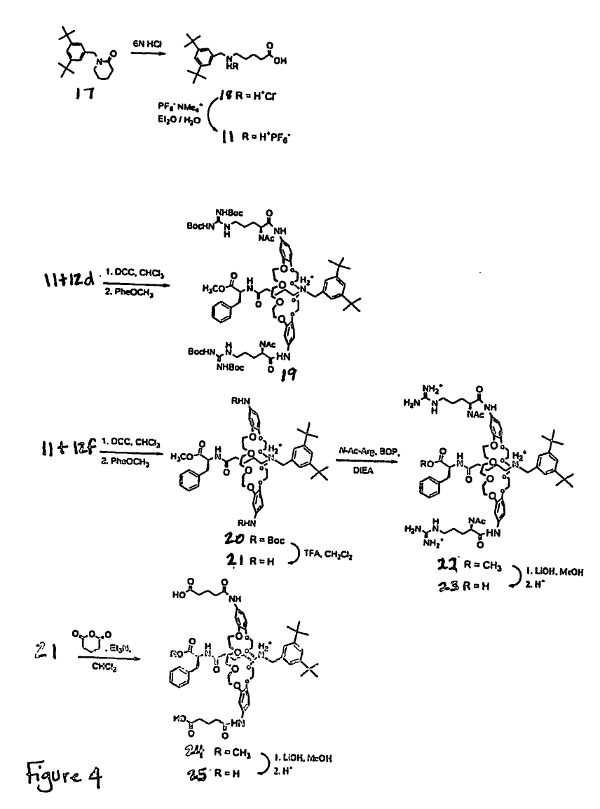

Referring to FIG. 4, an axle can be synthesized, for example, by acid hydrolysis of N-(Di-3,5-Di-tert-butylbenzyl)-δ-valerolactam (lactam) 17 to form 5-(3,5-Di-tert-butylbenzylamino) Pentanoic Acid Hydrochloride (amino acid) 18. Amino acid 18 forms axle component 11 after a counterion exchange of Cl⁻ with PF₆⁻ by adding PF₆⁻, Nme₄⁺, and Et₂O/H₂O.

In one aspect, as demonstrated by FIG. 4, a wheel component with at least one attached recognition element can be threaded onto axle 11. Such threading can occur, for example, in CHCl₃ by adding axle 11 and a derivatized wheel with attached Boc protecting group(s). The addition of DCC produces DCC-rotaxane 9. As an example, adding PheOMe and Et₃N to the DCC-rotaxane leads to the formation of rotaxane 19.

Alternatively, recognition elements can be attached to the wheel component after host-rotaxane formation. In such a case, a wheel component with an attached protecting group(s) can be threaded on the axle to form a host-rotaxane structure. Once the protecting groups are removed using any method known in the art, including those discussed herein, the desired recognition elements can be attached to the wheel component on the host-rotaxane structure. This synthesis methodology can be used, for example, when large recognition elements are attached to the wheel component. For example, as demonstrated in FIG. 4, axle 11 and, for example, crown ether 12f can be combined to form a rotaxane structure by adding DCC and CHCl₃, followed by PheOCH₃ to form Boc-protected phenylalanine methyl ester di(tert-butoxycarbonylaminobenzo)[24]crown-8 rotaxane 20. Rotaxane 20 can be deprotected using 30% TFA and CH₂CL₂ to form phenylalanine methyl ester di(aminobenzo)[24]crown-8 rotaxane 21. The arginine recognition elements can then be attached to the rotaxane structure by adding BOP activated N-acetylarginine in DIEA to fon-n phenylalanine methyl ester di(aminobenzo) [24]crown-8 rotaxane 22. If desired, ester hydrolysis can be performed using, for example, LiOH and MeOH followed by the addition of H⁺ to form phenylalanine di(N-acetylarginylaminobenzo)[24]crown-8 rotaxane 23.

As a further example, recognition element 12b can be added to rotaxane 21 by the addition of glutaric anhydride, Et₃N, and CHCl₃ to yield phenylalanine methyl ester di(4-carboxybutyrylaminobenzo)[24]crown-8 rotaxane 24. The addition of LiOH and MeOH, followed by the addition of H⁺ to rotaxane 24 yields phenylalanine di(4-carboxybutyrylaminobenzo)[24]crown-8 rotaxane 25.

The present disclosure further contemplates the manipulation of blocking groups to provide, for example, compounds that contain sufficient recognition elements to target and bind with an identified guest molecule or series of guest molecules. Such guest binding elements are, for example, calixarene, cleft, or cyclophanes, among others known in the art. Depending on the guest binding element, the methods of synthesis can be different, and will be discussed below.

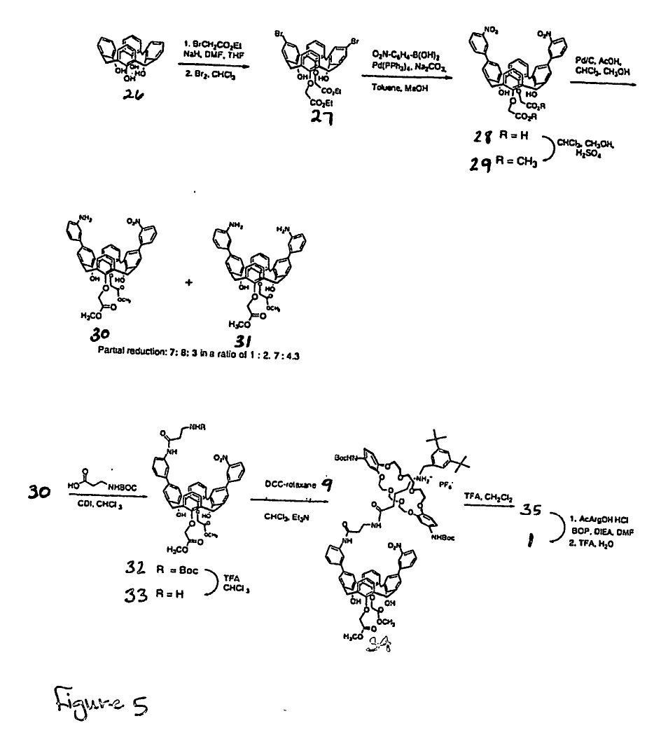

A synthesis scheme for calixarenes is, for example, depicted in FIG. 5. Calix[4]arene as a guest binding element can be synthesized, for example, by selective functionalization of calix[4]arenes by selective dialkylation of the lower rim of calix[4]arene 26, followed by electrophilic substitution of the phenolinic units of the dialkylated calix[4]arenes. The lower rim of the calix[4]arene 26 can be alkylated with ethyl bromoacetate (BrCH₂CO₂Et) in a mixture of TEF and DMF, followed by the addition of Br₂ and CHCl₃ to yield 5,17-dibromo-25,27-bis(ethoxycarbonylmethoxy)calix[4]arene-26,28-diol (calix[4]arene) 27. m-nitrophenyl rings can be successfully attached to calix[4]arene 27, for example, by a Suzuki coupling reaction of m-Nitrophenylboronic acid and calix[4]arene 27 using Na₂CO₃, H₂O, tolulene, and methanol, which hydrolyzes the ethyl esters. The acids can then be re-esterfied to form 5,17-bis(3-nitrophenyl)-25,27-bis(hydroxycarbonylmethoxy)calix[4]arene-26,28-diol (calix[4]arene) 28. Calix[4]arene 28, poorly soluble in most organic solvents, can be purified by trituration with ethyl ether to remove excess m-Nitrophenylboronic acid and then re-crystallized by methanol. Refluxing calix[4]arene 28 in a MeOH/CHCl₃ solution with a catalytic amount of acid forms 5,17-bis(3-nitrophenyl)-25,27-bis(methoxycarbonylmethoxy)calix[4]arene-26,28-diol (dinitrocalix[4]arene) 29.

In order to synthesize a host-rotaxane with calix[4]arene as a guest binding element, selective reduction of one of the two nitro groups on dinitrocalix[4]arene 29 is necessary. A conventional reduction method can be used, such as, for example, Pd/C; H₂, Pd/C, and HCO₂NH₄; or Pd/C, H₂ in CHCl₃ or in a CHCl₃/methanol solution. A preferred method of monoreduction, however, involves the addition of acetic or formic acid to a solution of dinitrocalix[4]arene 29 in a CHCl₃/methanol mixture (50/50 (v/v)) in the presence of Pd/C under a H₂ atmosphere. The reaction can be monitored by thin layer chromatography and terminated when approximately 70-80% of dinitrocalix[4]arene 29 is consumed, which usually occurs in approximately 12-18 hours. The crude reaction mixture can then be separated by column chromatography to give calix[4]arenes 29, 30 (5-(3-aminophenyl)-25,27-bis(methoxycarbonylmethoxy)calix[4]arene-26,28-diol), and 31 (5,17-bis(3-aminophenyl)-25,27-bis(methoxycarbonylmethoxy)calix[4]arene-26,28-diol) in yields of approximately 1, 2.7, and 4.3 ratios, respectively.

To couple calix[4]arene 30 to a rotaxane structure, the addition of a primary amine is necessary. This can be accomplished, for example, by attaching Boc-β-alanine using EEDQ in refluxing pyridine, or alternatively through CDI coupling in refluxing CHCl₃, which produces 5-[3-(3-tert-butoxycarbonylamino)-phenyl]-17-(3-aminophenyl)-25,27-bis(methoxyearbonylmethoxy)calix[4]arene-26,28-diol (Boc-β-alanylcalix[4]arene) 32. Boc-β-alanylcalix[4]arene 32 can then deprotected using 20% TFA in CH₂Cl₂ to give 5-[3-(3-aminopropionylamino)-phenyl]-17-(3-aminophenyl)-25,27-bis(methoxycarbonylmethoxy)calix[4]arene-26,28-diol (calix[4]arene) 33.

A calix[4]arene can be attached to a rotaxane structure, for example, by adding calix[4]arene 33 and DCC-rotaxane 9, which contains a Boc-protected wheel, to a solution of CHCl₃ and Et₃N to form di[(tert-butoxycarbonylamino)benzo]-[24]crown-8 host rotaxane 34. The wheel component can then be deprotected with exposure to TFA and CH₂Cl₂ to yield calix[4]arene di(aminobenzo)[24]crown-8 host rotaxane 35. A desired recognition element(s) can be coupled to the wheel using any of the previously discussed synthetic methodologies, or any others known in the art. For example, arginine-based recognition elements can be attached by first adding AcArgOH HCl, BOP, DIEA, and DMF, and then adding TFA and H₂O which yields calix[4]arene di[N-acetylarginylamino)benzo][24]-crown-8 host rotaxane (calixarene-rotaxane) 1.

Methods of synthesizing open cleft and cyclophane molecules are known in the art and are disclosed, for example, by Krieger, C., Deiderich, F. Chem. Ber. 1985, 118, 3620-3631, which is herein incorporated by reference. New methods of synthesis, however, have been created to attach a guest binding element (i.e., cyclophane or cleft) to a linear component of a host-rotaxane.

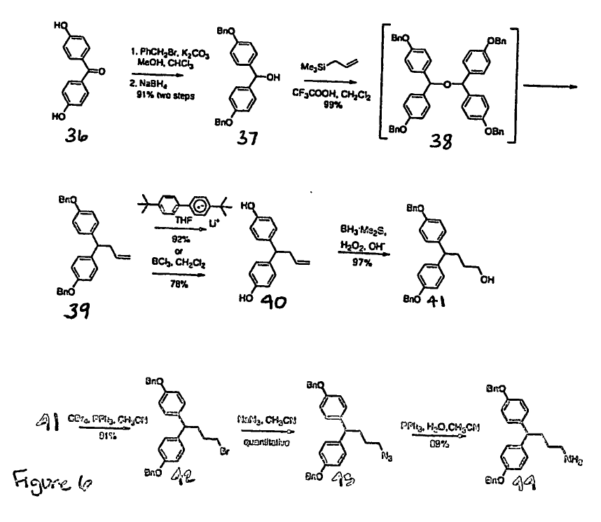

A scheme for the synthesis of cleft, is, for example, depicted in FIG. 6. The synthetic scheme can, for example, begin with 4,4′-Dihydroxybenzophenone 36 as a commercially available precursor of cleft or cyclophane. The phenolic oxygen atoms can then be protected as benzyl ethers by adding PhCH₂Br, K₂CO₃, MeOH, and CHCl₃. The carbonyl group can then be reduced to produce bis-(4-benzyloxyphenyl-methanol 37 by adding NaBH4. CF₃COOH (TFA) can then be added to cause the formation of a carbocation through dehydration of the secondary dibenzyl alcohol. Allyltrimethylsilane can further be added to react with the resultant carbocation to form a C—C bond, but the initial product is ether 38, which exists in equilibrium with the carbocation. After extended reaction time, for example, 24 hours, the carbocation is completely consumed through an irreversible reaction with allyltrimethylsilane to form 3,3-bis-(4-benzyloxyphenyl)-propene (cleft)₃₉. Deprotection of cleft 39 can occur by using, for example, lithium di-tert-butylbiphenylide and THF to produce 3,3-bis-(4-hydroxyphenyl)-propene (bisphenol) 40, which produces a high yield, but is a costly reagent. A more economical approach to deprotection of cleft 39 to form bisphenol 40 can be accomplished using BCl₃ with CH₂Cl₂, but does not result in as high a yield as the previously discussed deprotection method.

BH₃.Me₂S, H₂O₂, and OH⁻ can then be added to bisphenol 40 to form benzylcleft-alcohol 41, and then brominated by adding CBr₄, PPh₃, and CH₃CN to form cleft-bromide 42. The bromide on cleft-bromide 42 can then be replaced with azide by treating it with NaN₃ and CH₃CN to produce cleft-azide 43. Adding PPh₃, H₂O, and CH₃CN reduces the azide and forms cleft-amine 44.

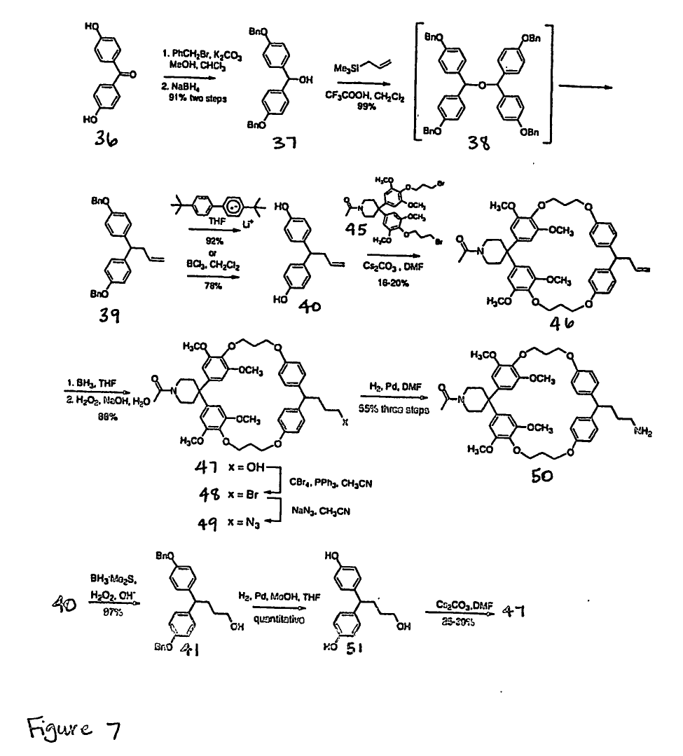

Referring to FIG. 7, the cyclophane synthesis pathway is similar to that of the cleft disclosed above, but deviates after the formation of bisphenol 40. After bisphenol 40 is formed, it can then be coupled with dibromide 45 in Cs₂CO₃ and DMF to form cyclophane-alkene 46. Cyclophane-alcohol 47 can then be formed by hydroboration of the olefin on cyclophane-alkene 46 by first treating the alcohol with BH₃ and THF and then adding H₂O₂, NaOH, and H₂O. Similar to the cleft synthesis previously discussed, cyclophane-alcohol 47 can then be brominated using CB₄, PPh₃, and CH₃CN. The resulting cyclophane-bromide 48 can then be displaced with azide by adding NaN₃ and CH₃CN to yield cyclophane-azide 49. The azidoalkane on cyclophane-azide 49 can then be reduced using H₂, Pd, and DMF to produce cyclophane-amine 50.

Alternatively, bisphenol 40 can be treated with BH₃.Me₂S, H₂O₂, and OH⁻ to form benzyl-cleft 41 prior to macrocyclization, which allows the routine H₂, Pd, MeOH, and THF reduction method to remove the benzyl groups to yield cleft-alcohol 51. The cleft-alcohol 51 can then be coupled with dibromide 45 in Cs₂CO₃ and DMF to form cyclophane-alcohol 47. This alternative synthesis route provides an improved yield of cyclophane 41 of 25-30% as compared to cyclophane 46, which has a yield of 16-20%.

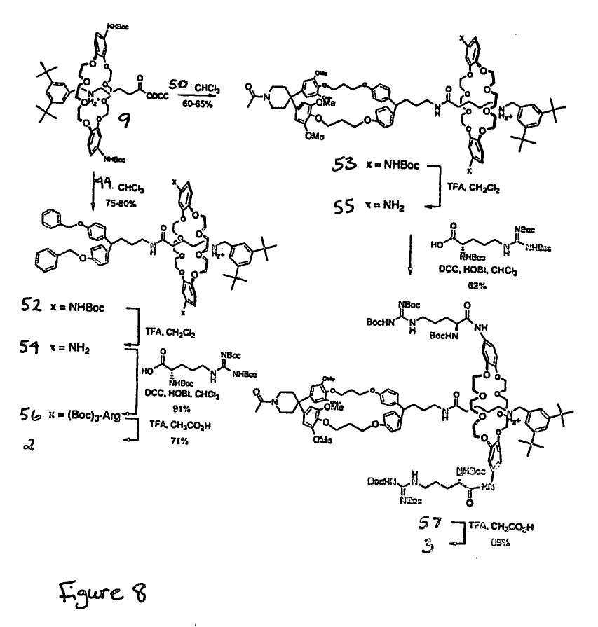

Referring now to FIG. 8, formation of host-rotaxanes can be accomplished by combining DCC-rotaxane 9, which is present as a mixture of syn and anti constitutional isomers (the syn isomer is shown in the drawings), with a nucleophile, such as, for example, a primary amine of a cleft, calixarene, or cyclophane. Cleft, for example, can be coupled to DCC-rotaxane 9 by adding cleft-amine 44 and CHCl₃ to a mixture of DCC-rotaxane 9 to yield 75-80% BocNH-cleft-[2]rotaxane 52. Cyclophane, for example, can be coupled to DCC-rotaxane 9 by combining a mixture of DCC-rotaxane 9 and cyclophane-amine 50 in CHCl₃ to form BocNH-cyclophane-[2]rotaxane 53 in 60-65% yields.

The addition of the functional groups on both cleft-[2]rotaxane 52 and cyclophane-[2]rotaxane 53 can occur using similar synthesis steps. Boc protecting groups on the wheel can be removed by adding TFA in CH₂Cl₂ to form either NH₂-cleft-[2]rotaxane 54 or NH₂-cyclophane-[2]rotaxane 55. Any desired functional group or recognition element can be added using any synthetic method known in the art, such as those previously discussed.

For example, arginine recognition elements can be attached to the wheel using fully Boc protected arginines through DCC coupling with a catalytic amount HOBt in CHCl₃ to form (Boc)₃-Arg-cleft-[2]rotaxane 56 or (Boc)₃-Arg-cyclophane-[2]rotaxane 57. The Boc protecting groups can be removed using a 1:1:1 ratio of TFA, CH₃CO₂H, and CH₂Cl₂ to form cleft-[2]rotaxane 2 or cyclophane-[2]rotaxane 3.

While the present invention has been illustrated by description of several embodiments, and while the embodiments have been described in considerable detail, it is not the intention of the applicant to restrict, or in any way limit the scope of the appended claims to such detail. Additional advantages and modifications may readily appear to those skilled in the art.

Methods of Using Host-rotaxanes

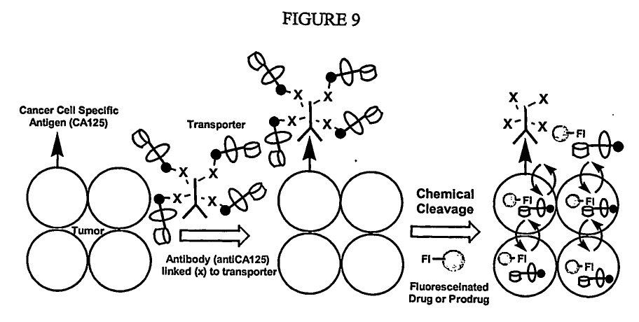

The present invention also provides for the Antibody Directed Cellular Transport (ADCT) method (FIG. 9). The Antibody Directed Cellular Transport method is designed to deliver drugs or prodrugs selectively into cells, cancer cells and solid tumors. Cell specific antibodies selectively bind their antigenic cellular target. Tagging these antibodies with a host-rotaxane composition via a cleavable linker will bring a high concentration of the host-rotaxane composition to the cell-surface. The link is cleaved once the composition reaches the cell or tumor (by enzymatic action or pH change or light activation). An administered fluorescein tagged drug will be delivered into the tumor by the host-rotaxane that resides in the tumor. Once inside the cell, the Fl-drug is released because of dilution or specific interactions with the drug's cellular target. Delivering an inhibitor of a cancer specific enzyme or a prodrug that is selectively triggered in a cancer cell would provide an additional level of selectivity. Once all the components arrive at the cell or tumor, the antibody may no longer be required. The host-rotaxane composition may prefer the tumor environment of the serum, constantly traveling throughout the cells, bringing Fl-drugs deep within the tumor. The host-rotaxane composition will be lodged into the tumor waiting for the drug or prodrug to appear.

Alternatively, the antibodies specific may have fluorescein (Fl) linked to their surfaces to provide a noncovalent attachment site for the host-rotaxane composition. Once the Fl-antibody-host-rotaxane composition is bound to the cell surface, a Fl-drug conjugate will be introduced. Swapping of binding partners will result in Fl-drug delivery and possible cell death.

One embodiment utilizes the Antibody Directed Enzyme Prodrug Therapy (ADEPT) method, which covalently links enzymes to antibodies. Prodrugs are introduced and become activated at tissues containing the antibody conjugate. The ADEPT method reduces unwanted toxic effects by activating a drug at targeted tissues. The ADCT method reduces toxicity by only transporting a drug into targeted cells. Thus, for the ADCT method, the drug is generally not cell permeable, whereas for the ADEPT method the drug is cell permeable. The advantage being there are more impermeable compounds than permeable ones. Bispecific linked antibodies have been used as the key recognition piece for bringing “killer cells” or other toxins to cancer cells. Cell penetrating liposomes have been guided to a particular cell by incorporating an antibody or a Fab fragment into the noncovalently assembled conglomerate. The ADCT method combines the advantages of ADEPT by bringing multiple agents to a targeted cell and liposomes by delivering nonpermeable drugs through noncovalent complexes with the additional potential advantage of delivering a greater than a stoichiometric amount of drug per transporter.

Specifically, the predicted promising features of the ADCT method are:
1) Selective targeting of cells. The target-binding moiety (antibody) of the ADCT method will play the same role as it does in the ADEPT method.
2) The host-rotaxane will act catalytically (traveling forward and back across the membrane multiple times) to deliver multiple drugs or prodrugs into the targeted cells. This activity is similar to the enzyme of the ADEPT method converting multiple prodrugs to drugs.
3) The immunogenicity of an antibody-host-rotaxane conjugate (with a ‘humanized’ Ab) may be less than the antibody-enzyme conjugate of the ADEPT method.
4) Antibody linkage will keep the host-rotaxane composition in the serum (host-rotaxane compositions are stable in fetal bovine serum for at least 1 week) and away from metabolic pathways of the various organs. The host-rotaxane compositions may prefer to reside within the tumor, further removed from the metabolic pathways.
5) The transport of prodrugs that have shown to react selectively in the reducing environment of the target cells (hypoxic cells) will provide an additional level of specificity besides antibody-antigen recognition. Another option, as an example, is the delivery of an inhibitor of a cancer specific enzyme.
6) If drug resistance occurs, an allergic reaction occurs, or a more appropriate drug is needed during therapy, a new drug can be linked to fluorescein and administered. The ADEPT method would require a new antibody-enzyme conjugate.
7) Once brought to the tumor surface, the host-rotaxane composition should reside selectively within the tumor (versus the serum) and deliver drugs deeply into the tumor.
8) Being readily designed and constructed, the host-rotaxane composition can be easily modified if necessary to improve the ADCT method, e.g., tumor association, drug association or transport ability.
9) The components of the ADCT method will be cheaper to produce than the ADEPT method (an enzyme does not need to be expressed and purified). The host-rotaxane composition can be made in high yields from cheap materials.
10)A wide variety of covalent bonds are known that are readily cleaved, for example, in weakly acidic media or by singlet oxygen.

The present invention also provides for a new approach to the delivery of low molecular weight compounds and peptides into eukaryotic cells using novel rotaxanes. Through noncovalent association, host-rotaxane 3 efficiently transports nonpermeable compounds, e.g., fluorescein-tagged oligopeptides (Fl-KKALRAQEAVDAL and Fl-KAASLWVGPR), a fluorescein-enzyme inhibitor conjugate, and fluorescein, at submicromolar concentrations into the cytoplasm and nucleus of eukaryotic COS-7 cells. Host-rotaxane 2 transports fluorescein.

Potential advantages of host-rotaxanes as drug transporters include, but are not limited to, (i) host-rotaxanes are small, which may make passive transport more likely; (ii) their construction involves a few synthetic steps using relatively cheap materials; (iii) they are serum stable in in vitro assays, however if unstable portions are found in animal studies, they can be easily replaced; (iv) the host-rotaxanes are well defined compounds, allowing them to be readily engineered to selectively recognize a drug or a drug conjugate; (v) a noncovalent carrier will not interfere with a drug's intracellular function; (vi) the host-rotaxane will travel back and forth through the membrane, delivering a greater than a stoichiometric amount of a drug; (vii) cell-targeting groups can be covalently or noncovalently attached; and (viii) host-rotaxanes will reduce the proteolysis rate of peptidic drugs. Synthetic pockets can protect biomolecules from degradation.

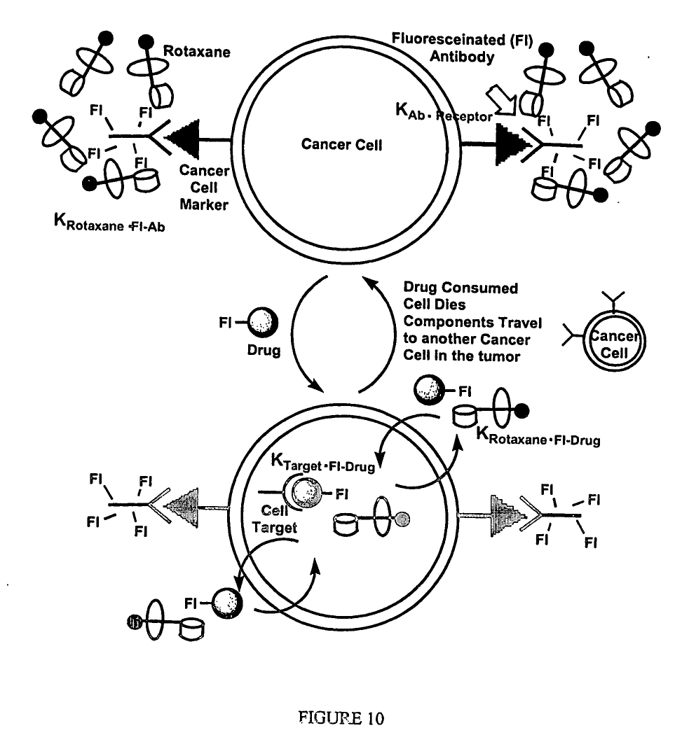

The ADCT method, illustrated in FIG. 10, is an easily adaptable method that connects the cell-selectivity provided by antibodies with nonpermeable anticancer drugs. Antibodies are an attractive targeting agent since cancer cells display unique and potentially antigenic groups on their surfaces. The specific interaction between an antibody and cell surface antigen is also used to bring a drug or prodrug to a cancerous cell.

Specifically, Antibody Directed Cellular Transport method can be accomplished with the rotaxanes being covalently or noncovalently linked to a targeting molecule, given as examples, antibody or peptide. Delivery depends on the magnitude of various association constants (K's) and the ability of cellular transporters to carry drugs through the membranes. Each association step allows selectivity. Cancer cell specific antibodies selectively bind their antigenic cellular target (K_{Ab-Receptor}) For the noncovalent ADCT method, antibodies will be tagged with fluorescein to provide an attachment site for cellular transporters (host-rotaxanes) (K_{Rotaxane-Fl-Ab}). For the covalent ADCT method, cellular transporters will be linked to the antibody by linkers that are engineered to break at the surface of cancer cells (light activation or changes in pH). Associating an antibody to its cell surface antigen brings a high concentration of transporters to the cell-surface. Introducing a fluorescein tagged drug to the cancer cell will cause the host-rotaxane to bind the Fl-drug and then deliver the drug into the cell. Once inside the cell the Fl-drug is released because of dilution or specific interactions with the drug's cellular target (K_{Target-Fl-Drug}).

Potential advantages of this method include, but are not limited to: 1) strong association between the antibody and antigen (generally K_{Ab-Receptor}=10⁹ M⁻¹) provides high selectivity, 2) multiple fluoresceins on the antibody surface (linked through Lys residues) provides an amplification of the number of cellular transporters, 3) metabolic stability provided by the antibody, 4) strong interactions between the host-rotaxanes and fluorescein (K_{A}=10⁵-10⁶ M⁻¹, phosphate buffer pH 7.0) keeps the Fl-antibody or Fl-drug associated in the concentrations used in the assay, 5) host-rotaxanes efficiently transport a divergent set of fluoresceinated compounds (peptides and PKC inhibitor at submicromolar concentrations), 6) various drugs can be fluoresceinated and transported, which is beneficial if drug resistance occurs, 7) cellular transporters can be made serum stable (carbon-carbon bonds, ether linkages, and peptidomimetic recognition elements), and thus, a catalytic amount of host-rotaxane can destroy multiple cancer cells and burry deep into a solid tumor.

Noncovalent or covalent attachment of the host-rotaxane to an antibody should decrease the rates of metabolic degradation and clearance. Protein binding is one of a myriad of factors that influence drug disposition. Renal excretion and hepatic metabolism are the predominate routes of drug elimination, and although metabolic stability is complex, drug elimination is greatly reduced by strong association of drugs (K_{A}=10⁵-10⁷ M⁻¹) to serum proteins. Clearance of even weakly associated drugs (K_{A}=10³-10⁵ M⁻¹) is slowed. The rotaxanes bind in the strongly associating range in buffer and moderately in serum. Association constants may differ in the body. For the ADEPT method, the antibody will be the serum binding protein. While not wishing to be bound by theory, the inventor believes that as long as the antibody stays in the serum the transporters will stay in the serum. Humanized antibodies have shown long half-lives in serum (e.g., t_{1/2}>1 week). Metabolic stability may also be obtained by the localization of the antibody and transporters at tumors and away from the major degradation pathways.

The host-rotaxanes may also be covalently linked to cell-targeting agents. As compared to the ADCT method, the targeting agent brings one equivalence of transporter to the cell surface, the covalent complex may not pass through the cellular membranes, and cell-entry may be receptor mediated and follow the endocytotic pathway. On the other hand, nonantibody-based targeting-agents may be cheaper to produce and be more biologically stable. Steroids fall in the last two categories, and being membrane-permeable, they may enhance the efficiency of transport. Furthermore, steroidal receptors exist, allowing those cells to be targeted. Interaction between 17β-estradiol and estrogen receptor (ER) plays an important role in breast carcinogenesis and breast cancer treatment. Estradiol has been used in combination with liposomes and small toxins to target cancer cells. In principle, the interaction between testosterone and prostate cells may be used as a target for prostate cancer therapies. The DCC-rotaxane method makes the addition of steroids to the transporters a relatively easy process. Small peptides are another attractive choice for a simple targeting agent. Using phage technology, researchers have discovered several peptides that interact selectively with cell surface proteins and antigens. The inventor has discovered that the host-rotaxanes deliver up to 1 3-mer peptides into cells. This size fits with the range of cell-targeting peptides that are being actively pursued. The DCC-rotaxane method also allows for the attachment of peptides to the transporters.

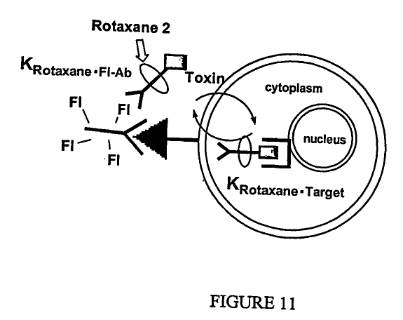

Successful transport of modified rotaxanes—covalently linked steroids or peptides—provide an alternative, ADCT method. Toxins are linked to the rotaxane, and these rotaxanes are selectively delivered to cancerous cells by Fl-antibodies. Delivery of the toxin-rotaxane into a cell requires breaking the rotaxane-Fl-antibody interaction (K_{Rotaxane-Fl-Ab}, FIG. 11). Thus, only a small amount of toxin-rotaxane may be available to kill a cell. On the other hand, if an equilibrium is established between the antibody-rotaxane complex (K_{Rotaxane-Fl-Ab}) and the rotaxane-target complex (K_{Rotaxane-Target}) results in substantial drug delivery in the case that K_{Rotaxane-Target}>K_{Rotaxane-Fl-Ab}. A variety of simple toxins, e.g., low molecular weight mustards, can be used since cell selectivity is accomplished by the antibody-antigen interaction. The toxin can be delivered to the cytoplasm or nucleus depending on the target location. An equilibrium established between the two binding domains and the rotaxane (K_{Rotaxane-Target} and K_{Rotaxane-Fl-Ab}) results in substantial drug delivery if K_{Rotaxane-Target}>K_{Rotaxane-Fl-Ab}.

Addition of Fl-antibody to a cell with an available antigen will have a green surface once the antibody-antigen complex is formed. Addition of the transporter and complexation to the fluorescein moiety of the Fl-antibody will result in fluorescence quenching. Addition of the Fl-drug will compete for the transporter and be delivered. Both the cell surface and interior will be green; color intensity depends on the degree of transporter association.

Linkers

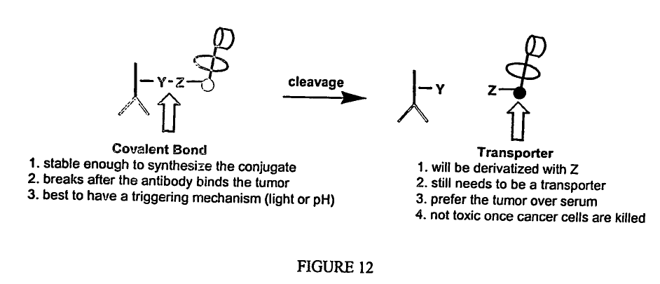

Create linkers that are cleaved in acidic media or by singlet oxygen. Having the transporters covalently linked to the guiding target-binding moiety (antibody) will ensure that most transporters reach the tumor. Selectivity and the pharmacokinetics of the conjugate will largely depend upon the target-binding moiety (antibody). Fortunately, there has been extensive research, clinical trials, and successes with therapeutic antibodies. The best linkers (FIG. 12) will cleave only at the tumor and not degrade over the time required for the antibody to reach the tumor (hours to days observed for the ADEPT method). Although using a linker will naturally modify the transporter, this additional functionality should not impede transport. We have shown that a fluorescein-linked model transporter (has the key pieces of the transporter) is still cell permeable (preliminary results).

(i) pH sensitive linkers. Low extracellular pH is a common feature of solid tumors (as low as pH 5.8), and this feature has been exploited in a few anticancer therapies. For the ADCT method, the best linker would be one that is stable in the serum (pH 7.4) and reacts at the tumor (pH=6). The required half-life in the serum depends on how fast the antibody reaches the tumor. For the ADEPT method, antibodies generally reach their targets within hours to days, depending on the pharmacokinetics of the conjugate. Keeping the transporter linked to the antibody will reduce unwanted delivery of the transporter into healthy tissues and should enhance its metabolic stability by keeping it in the serum. Half-life of the bond at the tumor should be significantly shorter. Preferably, a linkers are used with half lives of at least a two days in buffer at pH=7 and a couple of hours at pH=6. Fortunately, there has been extensive research into the hydrolysis rate of various covalent bonds, which have shown rates of a few minutes to months. These studies include exquisite examples of intramolecular catalysis and neighboring group participation, which provides unique opportunities to fine-tune the hydrolysis rate.

TABLE 1

| | half life | |
|---|---|---|
| 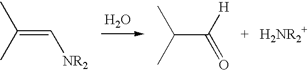 | pH = 7.5 Ph = 6 | 20 hr 0.2 hr |
| 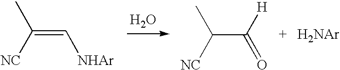 | pH = 7.5 pH = 6 | 360 hr 4 hr |
| 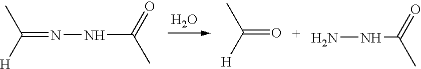 | pH = 7 pH = 5 | 40 hr 0.1 hr |

Covalent bonds that are dramaticly less stable in weakly acidic water. These bond types will be incorporated into linkers that will release the transporters upon Ab-transporter conjugate binding to solid tumors.

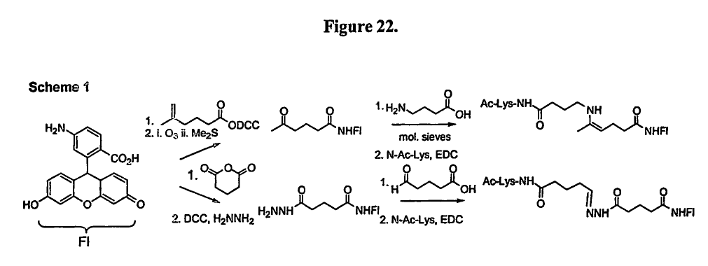

Enamine and acylhydrazone functional groups will be tested first. These functionalities have shown impressive differences in cleavage rates with changes in pH (Table 1) and are simple to form. The hydrolysis rate will be measured for linkers free in solution (via ¹H NMR analysis) and conjugated to an antibody (via fluorescence analysis). The linkers will have a fluorescein end in both studies to make the studies consistent and lessen the synthetic burden. The synthetic routes (FIG. 22) are straightforward, and we have made similar fluorescein derivatives. We have found at times that protecting fluorescein with Tosyl groups enhances their solubility in organic solvents, making synthesis easier. In the ADCT method, a transporter will replace fluorescein, and thus, it will carry either the ketone or hydrazine group. Either group should not interfere with transport. As mentioned, there is a wide range of cleavable covalent bonds that can be tested to fine-tune the hydrolysis rate using features such as steric hindrance and electronic properties. For example, note the difference in the hydrolysis rate for different substituted enamines (Table 1).

Methods for Measuring the Hydrolysis Rates. The linkers will be first tested without being linked to an antibody. To mimic antibody coupling, a protected lysine may be coupled to the free acid in buffered water (pH 7.5). This step will also show if extensive linker hydrolysis occurs during this step. The coupling reaction will be performed at 4° C. to slow the hydrolysis step. Note the hydrolysis half-life of the acylhydrazone shown in Table 1 at 4° C. would be approximately 2 weeks (using the known approximation that the rate is cut in half with a 10 degrees drop in temperature). The formation of hydrolyzed products in ¹H NMR spectra of the linkers dissolved in 90% buffer (pH 7.5, 7.0, 6.5, and 6.0; 10 mM phosphate buffer)/10% D₂O will be monitored over time. Plotting the amount of product formed over time and then calculating the slope of the line will determine hydrolysis rates.

Preferably, linkers (hydrolysis slow at pH 7.5 and fast at pH 6.0) will be covalently linked to antibody CA125 through EDC coupling at 0° C. and pH 7.5 (FIG. 22, with Ab replacing N-Ac-Lys). Excess linker will be removed by dialysis at 4° C. (3×20 min). A combination of fluorescence and uv/vis absorbance analyses will give the amount of fluorescein attached to the antibody. The antibody-linker conjugate will be placed in an Dialysis Cassette, which will be subsequently placed in dialysis buffer at a set pH value (7.5, 7.0, 6.5, and 6.0; 10 mM phosphate buffer) at 30° C. Quantifying the amount of fluorescence intensity loss in the tube-solution over time will provide the hydrolysis rate.

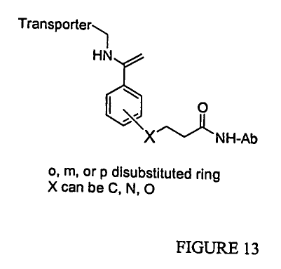

A variety of linkers can be constructed to fine-tune the hydrolysis rate. One linker is shown in FIG. 13. Changing linking orientation (o, m, or p) and the electronic property of the aromatic ring (X=C, N, or O) adjusts the hydrolysis rate at pH 7.5 and 6.0.

(ii) Light activated linkers. Photodynamic therapy involves the incorporation of a dye into a tumor that converts triplet oxygen to singlet oxygen upon long wave radiation (λₘₐₓ>600 nm; the deep skin penetration window). Singlet oxygen is lethal to cells. Problems with this therapy include selective dye incorporation into tumors. A variety of alkenes react with singlet oxygen to produce a dioxetane, which subsequently rearranges and breaks the bond. Breslow has demonstrated that a sensitizer can be used to cleavage a covalent bond in an intermolecular process. We propose to have the sensitizer covalently linked to a suitable alkene to give linker breakage in an intramolecular process. The sensitizer will be on the antibody end of the linker to not interfere with transport. Although hypoxia is found in tumor cells, especially deep in solid tumors, according to our hypothesis, the antibody-linker-transporter conjugate only needs to be active at the outmost surface of the tumor.

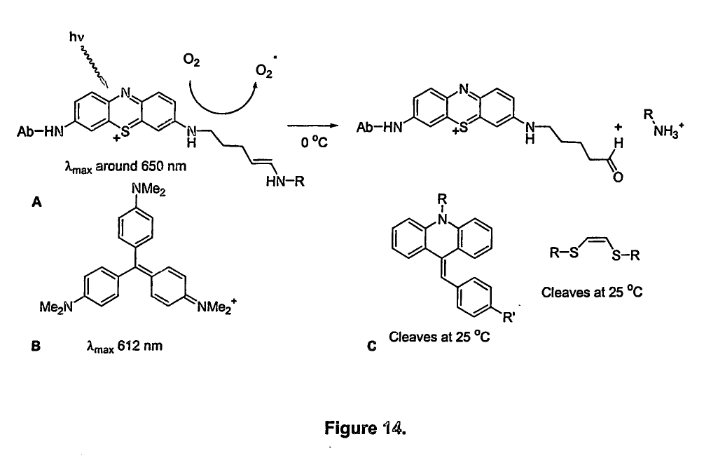

There are a variety of dyes that can be used, and many have the necessary difunctional groups to place the dye into a linker (e.g., FIG. 14B). We will first use thiazolium as the dye (FIG. 14A) because it is a small molecule and the two amino functional groups can be used for coupling. We have successfully derivatized acridine orange, which is structurally similar to thiazolium. The first active alkene will be an enamine since we will be synthesizing this moiety for the pH sensitive linker. Less hydrolyzable alkenes will be used as well (e.g., FIG. 14C).

Methods for Measuring the Cleaving Rates. The cleavage rate will be determined using the methods described above except that the dye will replace fluorescein and the rate of cleavage will be determined in the presence of light at 600 nm and without.

Preferably, these linkers will be stable in buffer for long periods of time (for drug storage) and only become activated at any desired time and body location using light activation.

In addition, rotaxanes can be made to specifically bind a different universal binding unit. For biodegradable linkers, a variety of prodrugs are made with groups, such as disulfides, which are reduced by glutathione, and esters, which are hydrolyzed by proteases, that degrade to give intracellular drug activation. Synthesis of fluorescein releasable compounds, should be relatively straightforward: various thioamines, thiocarboxylates, aminoalcohols, and anhydrides are commercially available and can be selectively protected if necessary.

Transport into Tumors

Referring now to Example 7-8, besides the increased acidity of their extracellular domains, tumors have limited and inefficient blood vessel networks, restricted and chaotic blood flow, and high variable interstitial pressures. These features make drug and prodrug penetration into solid tumors difficult. For the ADCT method, drug penetration will occur through transportation.

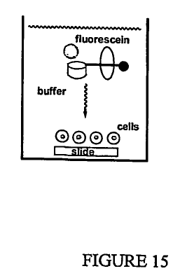

Without wishing to be bound be theory in any way, it is expected that the transporters will prefer to reside within the tumor as compared to the serum for the following reasons: (a) cell-transportation occurs when transporter and fluorescein are added separately to a 1 ml buffered solution in a well containing cells on a slide (FIG. 15); a crude model of blood and tumor, respectively; (b) the transporters are more soluble in organic solvents than water (water solubility is at least 0.1 mM pH 7 phosphate buffer); and (c) they deliver fluorescein into ovarian cancer cells (results not shown).

Summary of Binding Strength of Complexes

Bioactive compounds have a wide range of K_{D} values (e.g., the millimolar to nanomolar range). Strong drug-target interactions are not the sole requirement. Their absorption, distribution, metabolism, and excretion are just as important. For example, a weak complex may still be formed at a targeted site with a high local concentration of a drug.

Fluorescein is transported into eukaryotic COS-7 cells at a concentration of 4×10⁻⁷M with transporter 3 at a concentration of 6×10⁻⁷ M. At the start of the experiment, 40% of the available fluorescein is complexed, which means at the most 10⁻⁷ M of fluorescein is transported. These values were derived by assuming the association constant for the 1.fluorescein complex in the cellular solution is similar to the one measured by fluorescence quenching assays (K_{A}=10⁵ M⁻¹) in phosphate buffer. Tighter complexes can be formed with rotaxanes, which suggests that a lower concentration of components can be used to deliver drugs. Rotaxane 7 forms a tight complex with fluorescein (K_{A}=5×10⁶ M⁻¹). A 3×10⁻⁹ M concentration of 7-fluorescein complex would be transported for an assay solution containing 1×10⁻⁷ M solution of rotaxane 7 and 1×10⁻⁸ M solution of fluorescein under the same conditions considered above. As discussed in the proposal, rotaxane 7 may be a transporter.

Size/Complexity of Rotaxane

Another embodiment provides for a modified ADCT method whereby the transporter itself is the toxin. Considering a modified cleft-rotaxane, these compounds can be of reasonable molecular weights (ca. 2000 g/mol); potential rotaxane-based transporters can have weights as low as 1000 g/mol. Furthermore, biodegradable rotaxanes are also a possibility whereby bonds are cleaved after prolonged exposure to biosolutions or biodegrading agents such as enzymes. As an example, wheels with an amide bond could be susceptible to enzymatic cleavage releasing the degraded wheel from the axle. Drug application involves an intravenous injection of the antibody (Ab)-transporter conjugate followed by oral delivery of a drug. If the serum stability of the Ab and transporter is significant (humanized Ab's half life can be 1 week) only a single injection may be necessary. The drug can be orally introduced for a week or more. For the other methods, either the transporter or a transporter-drug conjugate would be intravenously induced. Therefore, the rotaxanes are an alternative approach to the very large liposomes used for delivery.

Alternative Methods for Selective Transport

In another embodiment, the present invention also provides for using one or more target-binding moieties that can be advantageously combined to form a derivatized rotaxane molecule, by use of one or more linkers that contain one or more cleavage sites, for administration to a subject (that is, a “treated subject”), where the target-binding moieties are capable to directing the rotaxanes to the target site and can be released by cleavage molecules, such as enzymes, present in the treated subject. The rotaxane molecules herein are designed in such a manner as to be cleavable into component parts, preferably, at a desired location in the treated subject to achieve a biological effect either at the site of cleavage or at a location close by. Cleavage of the rotaxane molecules may take place in a substantially confined area in the treated subject, such as in the gastrointestinal tract (“GI”), in synovial fluid, or inside a cell, for example, or cleavage may take place systemically, such as in the blood or other body fluids. Cleavage of the derivatized rotaxane molecules releases rotaxanes that are functional in the treated subject and capable to transporting an agent across cellular membranes. Such rotaxanes may or may not be active prior to cleavage from the target-binding moiety.

Thus, the present invention includes methods of delivering rotaxanes to a treated subject to achieve a biological effect therein by administering rotaxane molecules thereto, each rotaxane molecule containing at least one target-binding moiety, each of which are linked to another by a linker that contains one or more cleavage sites for cleavage by cleavage molecules in the treated subject. Further, it is not necessary for all the cleavage sites in the rotaxane molecules to be cleaved at the same time or completely. One or more target-binding moieties may be cleaved from the rotaxane molecule while other target-binding moieties remain as part of the remaining rotaxane molecule. As an example, the rotaxane molecule herein may bind to a tissue, such as an extracellular matrix, in an uncleaved or partially cleaved form, and rotaxanes may be released therefrom from time to time when a certain enzyme level at that location is high. In addition, the rotaxane delivery may be active as part of the rotaxane molecule without being cleaved as long as the active site of such molecule is free to interact with other agents.

The present invention includes rotaxane molecules that have cleavage sites that are designed for cleavage at a desired location in the treated subject. For example, the rotaxane molecule herein may be designed for cleaved by an enzyme in the GI tract of the treated subject to release rotaxane molecules for activities therein. In such an instance, the rotaxane molecule is constructed with a linker that has one or more cleavage sites for one or more enzymes in the GI tract, such as an enterokinase cleavage site, for example. The amino acid sequence representing the enterokinase recognition or cleavage site is known and is generally represented by the amino acid sequence: -Lys-Lys-Lys-Lys-Asp-. The rotaxane molecule with an enterokinase cleavage site can be made in any conventional manner known in the art.

The types of cleavage sites suitable for incorporation into the linkers of the present rotaxane, molecules include certain ones that can be cleaved by certain treated subject enzymes (hereafter, “target enzymes”). Starting with all proteases present in a treated subject, including those endogenous to the treated subject and those that may be introduced by infecting pathogens, the cleavage sites suitable for use herein exclude those that are substrates for amino and carboxy peptidases and exclude those that are non-specific. However, less specific endopeptidases, such as trypsins, chymotrypsins, and elastases, will find use herein. In one embodiment of the present invention, the cleavage sites include those that are substrates for endopeptidases. In an aspect of this invention, the cleavage sites suitable herein include those that are substrates for intracellular enzymes. In another aspect of the present invention, the cleavage sites include those that are substrates for extracellular enzymes. In a further aspect of the present invention, the cleavage sites include those that are substrates for enzymes that are active at a cell surface. Notably, the target enzymes are constitutively expressed or are inducible. They circulate either systemically or locally.

The present invention further includes rotaxane molecules having cleavage sites that are designed for intracellular cleavage in the treated subject. In one aspect of the invention, the cleavage site is designed for cleavage by an intracellular enzyme that is endogenous to the treated subject. In another aspect of the invention, the cleavage site is designed for cleavage by any enzyme present intracellularly in the treated subject, whether endogenous or not, provided that the rotaxane molecule is not a combination consisting of a transduction domain and a cytotoxic domain or that the second component molecule is not a cytotoxic molecule. In another aspect of the invention, the cleavage site is designed or engineered for cleavage intracellularly in the treated subject, provided that the cleavage site is not a pathogen activated cleavage site from a pathogen infecting the treated subject cell. Thus, for example the cleavage site of the present invention may be designed for an enzyme to be separately induced in or introduced into the treated subject.

The present invention also includes administration of rotaxane molecules having a structure as above but with cleavage sites that are designed for enzymatic cleavage extracellularly in the treated subject, regardless of whether the enzyme is endogenous to the subject or not, constitutively expressed in the subject or inducible in the subject. Extracellular cleavage can take place anywhere in the subject, such as, for example, in any body fluids, including but not limited to: lymph fluids, blood, synovial fluids, peritoneal fluids, spinal fluids, vaginal secretions and lung fluids. Extracellular cleavage can be cleavage on the surface of a cell. The present invention thus includes rotaxane molecules containing linkers with cleavage sites designed for enzymatic cleavage at a cell surface in a treated subject.

In light of the present invention, the selection of appropriate enzyme cleavage sites and sequences therefor, for use in the rotaxane molecules herein for cleavage at a desired location inside a treated subject is within the skill of a person in the art. Information regarding enzymes and their cleavage sites are available from numerous sources.

In some embodiments, the cleavage sites of the rotaxane molecules of the present invention includes not only those that are substrates for proteases, but includes those that are substrates for other enzymes, such as glycosidases and heparanases.

In another embodiment, the enzyme cleavage site or sites engineered into the rotaxane molecule are designed for enzymes that are expressed or heightened under disease, stress, pathogenic, allergic, premature birth or geriatric conditions, and other conditions requiring treatment.

The linker of the present invention includes those having one or more than one enzyme cleavage sites. The linkers herein can advantageously include a spacer molecule for example, so as to better expose the cleavage site to enzymes for cleavage. Thus, in one embodiment, the present invention includes a spacer in the linker to better expose the cleavage site to enzymatic action. In such instances, the linker can be a series of random amino acid residues that do not tend to fold upon themselves. These amino acid residues can thus be a chain of hydrophilic amino acid molecules, for example. Further, when a spacer is used, the present invention may optionally include the addition of another cleavage site in the linker such that the spacer may be cleaved together with the cleavage site to generate the appropriate active fragments.

In one aspect of the present invention, the linker herein optionally contains about 10 to 20 amino acid residues, more preferably about 11-17 amino acid residues (hereafter, a “spacer”).

In another embodiment of the present invention, the targeting moieties are antibodies or active fragments thereof (hereafter, “antibody components”). En one preferred embodiment, the antibody components are selected from a list of antibodies that have been approved by the FDA. Examples of such antibodies include, but are not limited to: anti-ILS, anti-CD11a, anti-ICAM-3, anti-CD80, anti-CD2, anti-CD3, anti-complement C5, anti-TNFα, anti-CD4, anti-α4β7, anti-CD40L (ligand), anti-VLA4, anti-CD64, anti-IL5, anti-IL4, anti-IgE, anti-CD23, anti-CD147, anti-CD25, anti-β2 integrin, anti-CD18, anti-TGFβ2, anti-Factor VII, anti-IIbIIa receptor, anti-PDGFβR, anti-F protein (from RSV), anti-gp120 (from HIV), anti-Hep B, anti-CMV, anti-CD14, anti-VEFG, anti-CA125 (ovarian cancer), anti-17-1 A (colorectal cell surface antigen), anti-anti-idiotypic GD3 epitope, anti-EGFR, anti-HBER2/neu; anti-αVβ3 integrin, anti-CD52, anti-CD33, anti-CD20, anti-CD22, anti-HLA, anti-TNF, and anti-HLA DR.

Newer agents for cell-recognition can be readily used with the host-rotaxane compositions. For example, small peptides have shown the ability to recognize specific cell types. Adding an enzyme to such a small peptide will probably alter its recognition ability to a much greater extent than if this peptide was covalently linked to the smaller sized fluorescein.

The transporters may be covalently linked to a cell-targeting steroid or a series of peptides. Steroids should not impede transport, and even may enhance it. Estradiol-rotaxanes should target breast cancer cells, and testosterone-rotaxanes should target prostate cancer cells.

(i) Rotaxanes with steroids as cell-targeting agents Testosterone and estradiol are recognized selectively by cytoplasmic receptors and delivered into the nucleus. Surface receptors or other proteins that give non-genomic responses may also exist.

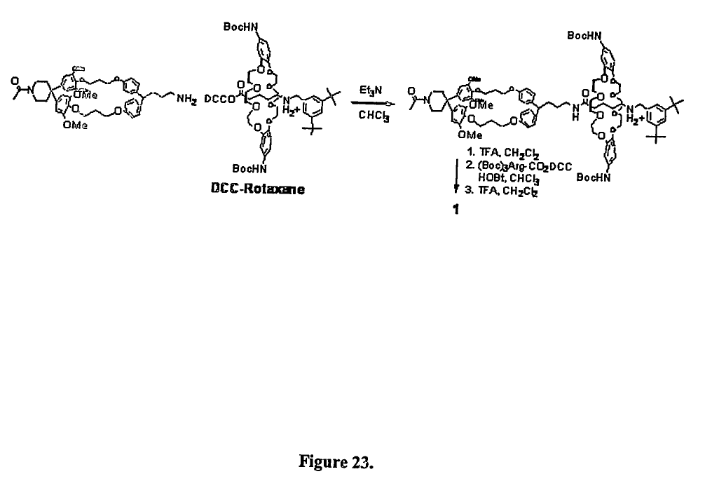

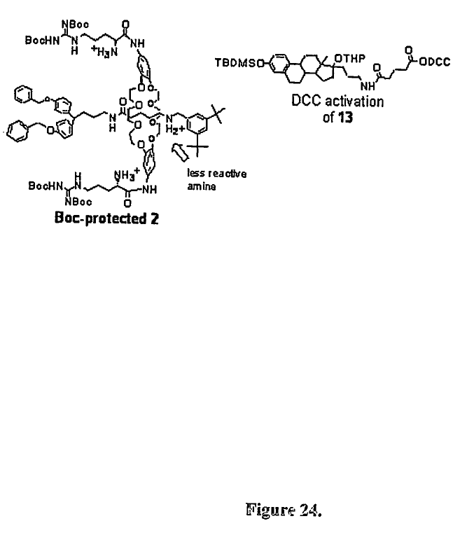

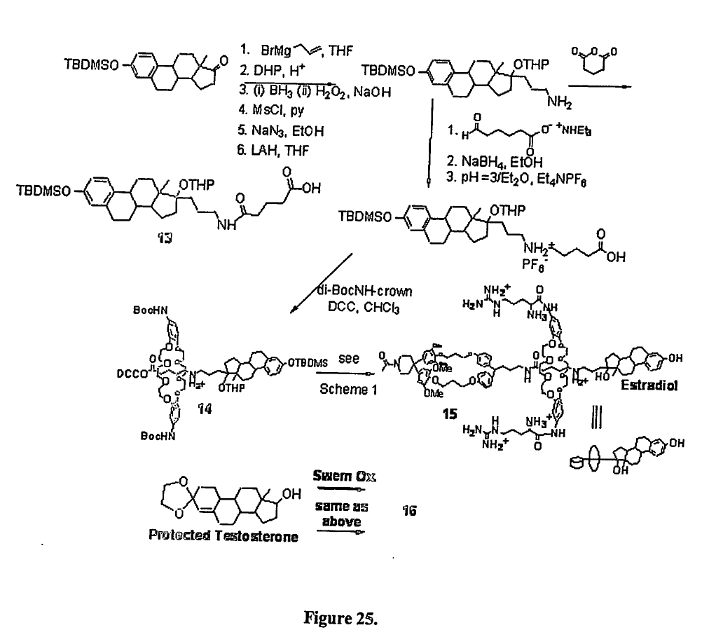

Synthesis of Steroid-Transporters There are three possible attachment sites: (i) the ring's amine (FIG. 24), (ii) the blocking group amine for rotaxanes made from DCC-rotaxane 6, and (iii) the blocking itself. In the latter case, the DCC-rotaxane method of rotaxane synthesis allows the relatively easy construction of novel steroid-transporters. Steroids (estradiol and testosterone) will be added to the recognition pocket through the reaction of DCC-rotaxanes, such as 14, with the amino host precursor (see FIG. 23) to give rotaxanes 15 and 16 (FIG. 25). Attachment at the C-17 carbon atom of the A-ring is desirable since the D-ring is a prerequisite for high affinity receptor binding for testosterone and the 3 and 17α hydroxyl groups are recognized by the ER. Another possible attachment site is at C-16.

Cellular Assays In contrast to the ADCT-method, these studies test the ability of the transporters to target intracellular receptors. While not wishing to be bound by theory, the steroid-transporters may deliver Fl-drugs to the nucleus to a greater extent than found for the ADCT-method.

The steroid-linked rotaxanes bound to fluorescein or a Fl-drug will be exposed to cells containing receptors for the steroids and ones without these receptors. Flow cytometric analysis will indicate the amount of compound delivered. A greater transport efficiency for cells with receptors will indicate selectivity. Excess steroid will be added to the assay solutions. If selectivity results from steroid-receptor interactions, the amount of compound transported will be reduced under these conditions. For example, rotaxane 15 and fluorescein will be added to breast cancer cells and to COS-7 cells. A greater amount of fluorescein delivered for estradiol-rotaxane 15 with breast cancer cells will indicate that cell selectivity occurs. This preference should be reduced with the addition of excess estradiol. Fluorescence microscopy experiments will show whether the steroid-transporters deliver compounds to the nucleus to a greater extent. Similar experiments will be performed with rotaxane 16, which is linked to testosterone and should result in prostrate cell selectivity.

(ii) Rotaxanes with peptides as cell-targeting agents. Using current biotechnology techniques, researchers have identified small targeting peptides, and many target various cancer cells. Small peptides have been added to delivery systems, such as liposomes, as cell-targeting agents. The AHNP peptide (derived as a mimic of the CDR3 loops of anti-p185 HER2/neu monoclonal antibodies) or the AntpHD peptide (a peptide vector that delivers the CTL epitope to antigen presenting cells) combined with liposomes bind their cellular targets. An APRPG-modified liposome, containing an anticancer drug, was used to target the angiogenic endothelium, resulting in tumor growth inhibition. The addition of an RGD containing peptide to a liposome successfully targeted the integrin GPIIb-IIIa on activated platelets.

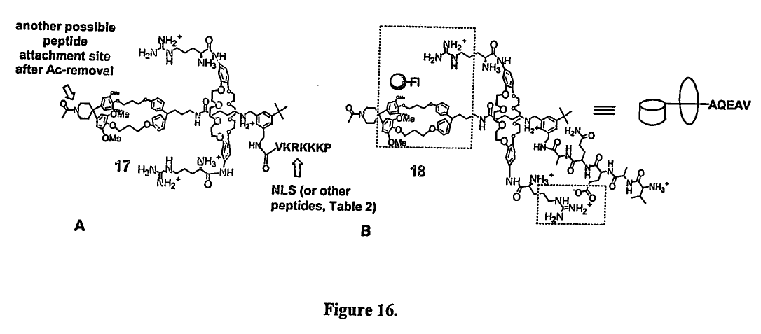

Model System for Peptide-Transporters One promising feature of the rotaxanes is the dual arginine residues on the ring. Molecular modeling results show that a single arginine residue interacts with the fluorescein. This suggests that the other arginine residue is available to interact with the attached peptide to cover any functional groups, e.g., carboxylates that impede membrane passage. For example, the AQEAV attached peptide of rotaxane 18 interacts with one arginine residue, whereas the other interacts with the carboxylate of fluorescein (this structure was a low energy structure in the molecular models, FIG. 16B).

TABLE 2
Pentapeptides chosen for transport

| Peptide^{a} | Side Chain Type^{b} |
|---|---|
| KKALR-CONH₂ | Cationic |
| AQEAV-CONH₂ | Anionic/Polar |
| AVDAL-CONH₂ | Anionic/Apolar |
| AQSAV-CONH₂ | Polar/Apolar |
| AVWAL-CONH₂ | Apolar |

^{a}Fl is fluorescein,
^{b}dominate side chains

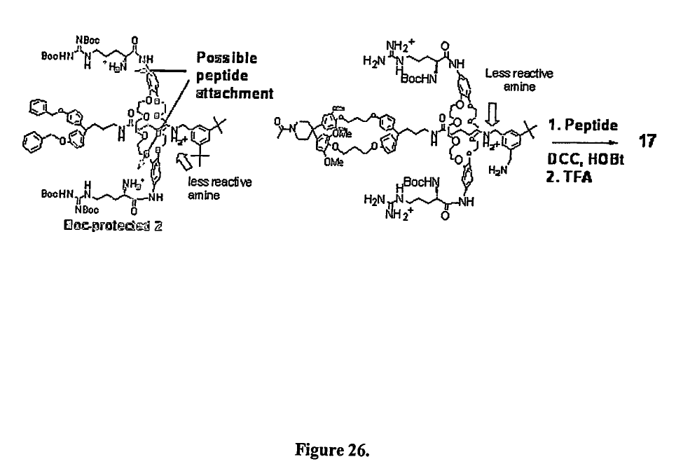

The pentapeptides shown in Table 2 will be attached to the amino group of a rotaxane's blocking group or the ring's amine (FIG. 16A; FIG. 26). These peptides are based on KKALRAQEAVDAL, which is transported by transporter 3 into COS-7 cells, and designed to highlight a certain type of side chain (cationic, anionic, polar, or apolar). Examining the transport efficiencies of these peptide-rotaxanes will show which types of peptide can be used as targeting agents. One may also attach the short SV-40 nucleus localizing factor PKKKRKV to a blocking group (rotaxane 17) to enhance transport into the nucleus. Western blot analysis of fixed cells will indicate the amount of material transported to the nucleus.

Cancer Specific Peptide-Transporters Once transport has been demonstrated, peptides that target cancer cells will be attached to the transporter. For example, peptides based on CVFXXXYXXC were found to bind the prostate-specific antigen (secreted enzyme) through the screening of phage libraries. CVFTSDYAFC has a K_{D} of 8 μM for PSA and will be added to the transporter. Selectivity will be demonstrated if the transporter delivers Fl-compounds to a greater extent into prostrate cancer cells verses other cell types. Selectivity will be verified if the addition of excess CVFTSDYAFC eliminates the observed cell-selectivity. We will also determine the transport mechanism. Many other cancer cell specific peptides can be attached to the rotaxanes.

Mechanism of Transport. One potential advantage of the ADCT method is that a greater than stoichiometric amount of Fl-drug can be delivered into the cell per transporter. This feat requires the transporter to pass through the membrane alone. The transporter should be permeable. It is composed of hydrophobic moieties and arginine (which can pass through membranes as part of peptides) and model rotaxanes are permeable throughout COS-7 cells (not shown). One caveat to this experiment is that the transporter would be modified with fluorescein. However, if transporters linked to a fluorescein moiety with an exposed carboxylate enters cells, then a transporter without a fluorescein moiety should enter cells. Other fluorophores, such as a coumarin, which are cell-permeable, can be attached.

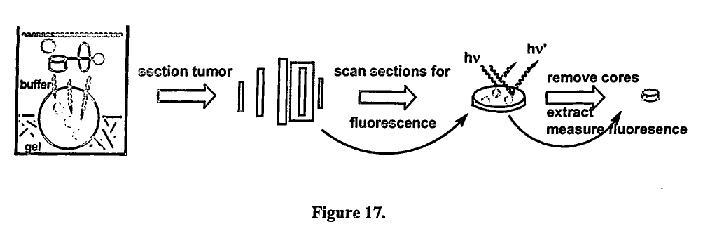

Biological Stability Assays. We have shown that a simple extraction procedure followed by HPLC analysis demonstrates that rotaxanes 1 and 2 are stable to fetal bovine serum at least for 6 days (FIG. 17). The transporters are designed to be serum stable. They are made from bisphenol A moieties, which are used in restorative dentistry, and have ether linkages, which have shown significant biological stabilities. Metabolism by the various P-450 enzymes is a possibility. However, associating the transporters to an antibody will keep them protected from metabolism by the liver until the antibody degrades (humanized antibodies have improved stability, e.g. t_{1/2}>1 week). While not wishing to be bound by theory, the transporters may be more soluble in the tumor mass, which should slow their metabolism. The same effect may be observed for rotaxanes that have steroids or peptides as targeting agents.

Biological stability will also be estimated by exposing the transporters to isolated enzymes. If amino acid hydrolysis occurs, the arginines (or other amino acid based recognition elements) will be swapped with peptidomimetics. The simplest alternative would be to use an alkyl chain containing a guanidinium group on its end.

Other Active Agents

Chemotherapeutics useful as active agents are typically small chemical entities produced by chemical synthesis. Chemotherapeutics include cytotoxic and cytostatic drugs. Chemotherapeutics can include those that have other effects on cells such as reversal of the transformed state to a differentiated state or those that inhibit cell replication. Exemplary chemotherapeutic agents include, but are not limited to, anti-tumor drugs, cytokines, anti-metabolites, alkylating agents, hormones, and the like.

Additional examples of chemotherapeutics include common cytotoxic or cytostatic drugs such as for example: methotrexate (amethopterin), doxorubicin (adrimycin), daunorubicin, cytosine arabinoside, etoposide, 5-4 fluorouracil, melphalan, chlorambucil, and other nitrogen mustards (e.g. cyclophosphamide), cis-platinum, vindesine (and other vinca alkaloids), mitomycin and bleomycin. Other chemotherapeutics include: purothionin (barley flour oligopeptide), macromomycin, 1,4-benzoquinone derivatives, trenimon, steroids, aminopterin, anthracyclines, demecolcine, etoposide, mithramycin, doxorubicin, daunomycin, vinblastine, neocarzinostatin, macromycin, α-amanitin and the like. Certainly, the use of combinations of chemotherapeutic agents is also provided.

Toxins are useful as active agents. Toxins are generally complex toxic products of various organisms including bacteria, plants, etc. Exemplary toxins include, but are not limited to, coagulants such as Russell's Viper Venom, activated Factor IX, activated Factor X or thrombin; and cell surface lytic agents such as phospholipase C, (Flckinger & Trost, Eu. J. Cancer 12(2):159-60 (1976)) or cobra venom factor (CVF) (Togel & Muller-Eberhard, Anal. Biochem 118(2):262-268 (1981)) which should lyse neoplastic cells directly. Additional examples of toxins include but are not limited to: ricin, ricin A chain (ricin toxin), Pseudomonas exotoxin (PE), diphtheria toxin (DT), bovine pancreatic ribonuclease (BPR), pokeweed antiviral protein (PAP), abrin, abrin A chain (abrin toxin), gelonin (GEL), saporin (SAP), modeccin, viscumin and volkensin.

Exemplary radiotherapeutic agents include, but are not limited to, 47Sc, 67Cu, 90Y, 109Pd, 123I, 125I, 131I, 111In, 186Re, 188Re, 199Au, 211At, 212Pb and 212Bi. Other radionuclides which have been used by those having ordinary skill in the art include: 32 P, and 33P, 71Ge, 77As, 103Pb, 105Rh, 111Ag, 119Sb, 121Sn, 131Cs, 143Pr, 161Tb, 177Lu, 1910s, 193 MPt, 197Hg, all beta negative and/or auger emitters. Some preferred radionuclides include: 90Y, 131I, 211At and 212Pb/212Bi.

Radiosensitizing agents are substances that increase the sensitivity of cells to radiation. Exemplary radiosensitizing agents include, but are not limited to, nitroimidazoles, metronidazole and misonidazole (see DeVita, V. T. Jr. in Harrison's Principles of Internal Medicine, p. 68, McGraw-Hill Book Co., N.Y. 1983, which is incorporated herein by reference), as well as art-recognized boron-neutron capture and uranium capture systems. See, e.g., Gabe, D. Radiotherapy & Oncology 30:199-205 (1994); Hainfeld, J. Proc. Natl. Acad. Sci. USA 89:11064-11068 (1992). A delivery rotaxane comprising a radiosensitizing agent as the active moiety is administered and localizes at the target tissue. Upon exposure of the tissue to radiation, the radiosensitizing agent is “excited” and causes the death of the cell.

Radiosensitizing agents are also substances that become more toxic to a cell after exposure of the cell to ionizing radiation. In this case, DNA protein kinase (PK) inhibitors, such as R106 and R116 (ICOS, Inc.); tyrosine kinase inhibitors, such as SU5416 and SU6668 (Sugen Inc.); and inhibitors of DNA repair enzymes comprise examples.

Exemplary imaging agents include, but are not limited to, paramagnetic, radioactive and fluorogenic ions. Preferably, the imaging agent comprises a radioactive imaging agent. Exemplary radioactive imaging agents include, but are not limited to, gamma-emitters, positron-emitters and x-ray-emitters. Particular radioactive imaging agents include, but are not limited to, 43K, 52Fe, 57Co, 67Cu, 67Ga, 68Ga, 77Br, 81Rb/81MKr, 87 mSr, 99 mTc, 111In, 113In, 123I, 125I, 127Cs, 129Cs, 131I, 132I, 197Hg, 203Pb and 206Bi. Other radioactive imaging agents known by one skilled in the art can be used as well.

In preferred embodiments of the invention, rotaxanes are targeted to tumor cells by conjugating antibody fragments to the rotaxane. Antibody targets that are overexpressed by tumors include, for example, CPSF, EphA3, G250/MN/CAIX, HER-2/neu, Intestinal carboxyl esterase, alpha-fetoprotein, M-CSF, MUC1, p53, PRAME, RAGE-1, RU2AS, Telomerase, WT1, among many others known in the art. In addition, antigens that are uniquely expressed by tumors are also suitable targets for antibodies. Such antigens include, for example, BAGE-1, GAGE-1 through 8, GnTV, HERV-K-MEL, LAGE-1, MAGE-1 through 12, NY-ESO-1/LAGE-2, SSX-2, TRP2/INT2 and others known in the art. The generation of monoclonal antibodies against any of these or other suitable targets is performed by methods, such as hybridoma technology, that are well known in the art. Isolation of antibody fragments, such as Fab′, or F(ab)₂, is a matter of routine for a person of skill in the art and can be performed by using published protocols such as those found in Harlow and Lane, Antibodies, A Laboratory Manual, Cold Spring Harbor Laboratory, (1988).

Dosages for Active Agents

For therapeutic applications, a therapeutically effective amount of a composition of the invention is administered to a subject. A “therapeutically effective amount” is an amount of the therapeutic composition sufficient to produce a measurable biological response (including, but not limited to an immunostimulatory response, an anti-angiogenic response, a cytotoxic response, or tumor regression). Actual dosage levels of active ingredients in a therapeutic composition of the invention can be varied so as to administer an amount of the active compound(s) that is effective to achieve the desired therapeutic response for a particular subject and/or application. The selected dosage level will depend upon a variety of factors including, but not limited to the activity of the therapeutic composition, formulation, the route of administration, combination with other drugs or treatments, severity of the condition being treated (e.g., in the case of a tumor, tumor size and longevity), and the physical condition and prior medical history of the subject being treated. In one embodiment, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine.

For diagnostic applications, a detectable amount of a composition of the invention is administered to a subject. A “detectable amount”, as used herein to refer to a diagnostic composition, refers to a dose of such a composition that the presence of the composition can be determined in vivo or in vitro. A detectable amount will vary according to a variety of factors, including, but not limited to chemical features of the drug being labeled, the detectable label, labeling methods, the method of imaging and parameters related thereto, metabolism of the labeled drug in the subject, the stability of the label (e.g. the half-life of a radionuclide label), the time elapsed following administration of the drug and/or labeled antibody prior to imaging, the route of drug administration, and the physical condition and prior medical history of the subject. Thus, a detectable amount can vary and can be tailored to a particular application. After study of the present disclosure, it is within the skill of one in the art to determine such a detectable amount.

Because delivery rotaxanes are specifically targeted to target tissues, a composition that comprises an active agent is typically administered in a dose less than that which is used when the active agent is administered directly to a subject, preferably in doses that contain up to about 100 times less active agent. In some embodiments, compositions that comprise an active agent are administered in doses that contain about 10 to about 100 times less active agent as an active moiety than the dosage of active agent administered directly. To determine the appropriate dose, the amount of compound is preferably measured in moles instead of by weight. In that way, the variable weight of delivery vehicles does not affect the calculation.

Typically, chemotherapeutic conjugates are administered intravenously in multiple divided doses. Up to 20 gm IV/dose of methotrexate is typically administered. When methotrexate is administered as the active moiety in a delivery composition of the invention, there is about a 10- to 100-fold dose reduction. Thus, presuming each delivery rotaxane includes one molecule of methotrexate to one mole of delivery rotaxane, of the total amount of delivery rotaxane active agent administered, up to about 0.2 to about 2.0 g of methotrexate is present and therefore administered. In some embodiments, of the total amount of delivery rotaxane/active agent administered, up to about 200 mg to about 2 g of methotrexate is present and therefore administered.

By way of further example, doxorubicin and daunorubicin each weigh about 535. Presuming each delivery rotaxane includes one molecule of doxorubicin or daunorubicin to one delivery rotaxane, a provided dose range for delivery rotaxane-doxorubicin vehicle or delivery rotaxane-daunorubicin is between about 40 to about 4000 mg. In some embodiments, dosages of about 100 to about 1000 mg of delivery rotaxane-doxorubicin or delivery rotaxane-daunorubicin are administered. In some embodiments, dosages of about 200 to about 600 mg of delivery rotaxane-doxorubicin or delivery rotaxane-daunorubicin are administered.

Toxin-containing loaded delivery rotaxanes are formulated for intravenous administration. Using an intravenous approach, up to 6 nanomoles/kg of body weight of toxin alone have been administered as a single dose with marked therapeutic effects in patients with melanoma (Spitler L. E., et al. (1987) Cancer Res. 47:1717). In some embodiments of the present invention, then, up to about 11 micrograms of delivery rotaxane-toxin/kg of body weight may be administered for therapy.

The molecular weight of ricin toxin A chain is 32,000. Thus, for example, presuming each delivery rotaxane includes one molecule of ricin toxin A chain to one delivery rotaxane, delivery rotaxanes comprising ricin toxin A chain are administered in doses in which the proportion by weight of ricin toxin A chain is about 1 to about 500 μg of the total weight of the administered dose. In some preferred embodiments, delivery rotaxanes comprising ricin toxin A chain are administered in doses in which the proportion by weight of ricin toxin A chain is about 10 to about 100 μg of the total weight of the administered dose. In some preferred embodiments, delivery rotaxanes comprising ricin toxin A chain are administered in doses in which the proportion by weight of ricin toxin A chain is about 2 to about 50 μg of the total weight of the administered dose.

The molecular weight of diphtheria toxin A chain is 66,600. Thus, presuming each delivery rotaxane includes one molecule of diphtheria toxin A chain to one delivery rotaxane, delivery rotaxanes comprising diphtheria toxin A chain are administered in doses in which the proportion by weight of diphtheria toxin A chain is about 1 to about 500 μg of the total weight of the administered dose. In some preferred embodiments, delivery rotaxanes comprising diphtheria toxin A chain are administered in doses in which the proportion by weight of diphtheria toxin A chain is about 10 to about 100 μg of the total weight of the administered dose. In some preferred embodiments, delivery rotaxanes comprising diphtheria toxin A chain are administered in doses in which the proportion by weight of diphtheria toxin A chain is about 40 to about 80 μg of the total weight of the administered dose.

The molecular weight of Pseudomonas exotoxin is 22,000. Thus, presuming each delivery rotaxane includes one molecule of Pseudomonas exotoxin to one delivery rotaxane, delivery rotaxanes comprising Pseudomonas exotoxin are administered in doses in which the proportion by weight of Pseudomonas exotoxin is about 0.01 to about 100 μg of the total weight of the loaded delivery rotaxane-exotoxin administered. In some preferred embodiments, delivery rotaxanes comprising Pseudomonas exotoxin are administered in doses in which the proportion by weight of Pseudomonas exotoxin is about 0.1 to about 10 μg of the total weight of the administered dose. In some embodiments, delivery rotaxanes comprising Pseudomonas exotoxin are administered in doses in which the proportion by weight of Pseudomonas exotoxin is about 0.3 to about 2.2 μg of the total weight of the administered dose.

To dose delivery rotaxanes comprising radioisotopes in pharmaceutical compositions useful as imaging agents, it is presumed that each delivery rotaxane is loaded with one radioactive active moiety. The amount of radioisotope to be administered is dependent upon the radioisotope. Those having ordinary skill in the art can readily formulate the amount of delivery rotaxane-imaging agent to be administered based upon the specific activity and energy of a given radionuclide used as an active moiety. Typically, about 0.1 to about 100 millicuries per dose of imaging agent, about 1 to about 10 millicuries, or about 2 to about 5 millicuries are administered.

Thus, compositions that are useful imaging agents comprise delivery rotaxanes comprising a radioactive moiety in an amount ranging from about 0.1 to about 100 millicuries, in some embodiments about 1 to about 10 millicuries, in some embodiments about 2 to about 5 millicuries, in some embodiments about 1 to about 5 millicuries.

To load delivery rotaxanes with radioisotopes in compositions useful as therapeutic agents, it is presumed that each delivery rotaxane is loaded with one radioactive active moiety. The amount of radioisotope to be administered is dependent upon the radioisotope. Those having ordinary skill in the art can readily formulate the amount of delivery rotaxane-radio-therapeutic agent to be administered based upon the specific activity and energy of a given radionuclide used as an active moiety.

Pharmaceutically Acceptable Formulations

After a sufficiently purified delivery rotaxane comprising active agent has been prepared, one will desire to prepare it into a pharmaceutically acceptable formulation that can be administered in any suitable manner. Preferred administration techniques include parenteral administration, intravenous administration and injection and/or infusion directly into a target tissue, such as a solid tumor or other neoplastic tissue. This is done by using for the last purification step a pharmaceutically acceptable medium.

Representative compositions generally comprise an amount of the desired delivery rotaxane-active agent in accordance with the dosage information set forth above admixed with an acceptable pharmaceutical diluent or excipient, such as a sterile aqueous solution, to give an appropriate final concentration in accordance with the dosage information set forth above with respect to the active agent. Such formulations will typically include buffers such as phosphate buffered saline (PBS), or additional additives such as pharmaceutical excipients, stabilizing agents such as BSA or HSA, or salts such as sodium chloride.

For parenteral administration it is generally desirable to further render such compositions pharmaceutically acceptable by insuring their sterility, non-immunogenicity and non-pyrogenicity. Such techniques are generally well known in the art as exemplified by Remington's Pharmaceutical Sciences, 16th Ed. Mack Publishing Company (1980), incorporated herein by reference. It should be appreciated that endotoxin contamination should be kept minimally at a safe level, for example, less that 0.5 ng/mg protein. Moreover, for human administration, preparations should meet sterility, pyrogenicity, general safety and purity standards as required by FDA Office of Biological Standards.

The pharmaceutical compositions encompassed by the invention may be administered by any means known in the art including, but not limited to oral or parenteral routes, including intravenous, intramuscular, intraperitoneal, subcutaneous, transdermal, airway (aerosol), rectal, vaginal and topical (including buccal and sublingual) administration. In preferred embodiments, the pharmaceutical compositions are administered by intravenous or intraparenteral infusion or injection.

For oral administration, the rotaxanes useful in the invention will generally be provided in the form of tablets or capsules, as a powder or granules, or as an aqueous solution or suspension.

Tablets for oral use may include the active ingredients mixed with pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while cornstarch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Capsules for oral use include hard gelatin capsules in which the active ingredient is mixed with a solid diluent, and soft gelatin capsules wherein the active ingredients is mixed with water or an oil such as peanut oil, liquid paraffin or olive oil.

For intramuscular, intraperitoneal, subcutaneous and intravenous use, the pharmaceutical compositions of the invention will generally be provided in sterile aqueous solutions or suspensions, buffered to an appropriate pH and isotonicity. Suitable aqueous vehicles include Ringer's solution and isotonic sodium chloride. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

The pharmaceutical compositions useful according to the invention also include encapsulated formulations to protect the rotaxane against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811; PCT publication WO 91/06309; and European patent publication EP-A-43075, which are incorporated by reference herein.

In one embodiment, the encapsulated formulation comprises a viral coat protein. In this embodiment, the rotaxane-containing formulation may be bound to, associated with, or enclosed by at least one viral coat protein. The viral coat protein may be derived from or associated with a virus, such as a polyoma virus, or it may be partially or entirely artificial. For example, the coat protein may be a Virus Protein 1 and/or Virus Protein 2 of the polyoma virus, or a derivative thereof.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred.

The data obtained from cell culture assays and animal studies can be used in formulation a range of dosage for use in humans. The dosage of compositions of the invention lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of the polypeptide) that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In addition to their administration individually or as a plurality, as discussed above, the rotaxanes useful according to the invention can be administered in combination with other known agents effective in treatment of diseases. In any event, the administering physician can adjust the amount and timing of rotaxane administration on the basis of results observed using standard measures of efficacy known in the art or described herein.

### EXAMPLES

### Example 1

Host-[2]rotaxanes are easily constructed using our DCC-[2]rotaxane method (FIG. 23). The unique architecture of rotaxanes, composed of interchangeable parts: axle, hosts, and blocking groups, and ring, (see compound 7) reduces the synthetic burden of creating many compounds. Although the ring exists as a mixture of syn and anti isomers, molecular modeling results show that both isomers bind guests equivalently. Single isomers are being synthesized.

The high yielding and straightforward route by swapping various blocking groups, allows the attachment of cell-targeting groups (steroids and peptides) and fluorophores. The addition of more biologically stable recognition elements, e.g., alkyl guanidine instead or arginine, can be accomplished by adding (Boc)₃-guanidine-(CH₂)₃—CO₂DCC or other activated peptidomimetics in step two of FIG. 23.

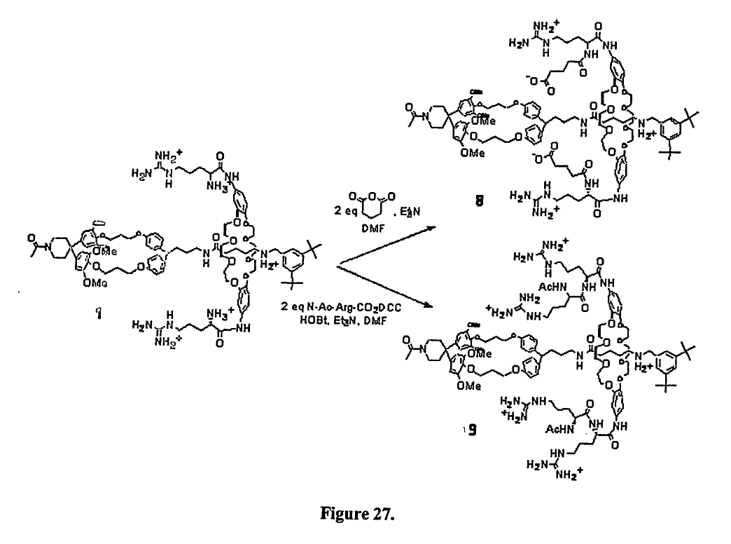

The DCC-rotaxane method allows the combination of various binding pockets or clefts and the easy attachment of recognition elements to the ring. For transporter 3, the primary amines of the arginine moieties are available for attaching other recognition elements (FIG. 27). We have just attached a carboxylate moiety to give rotaxane 8 and tetra-arginine rotaxane 9 will be constructed next.

### Example 2

Host-[2]rotaxane 1 was designed to selectively bind large aromatic acids, such as fluorescein. It binds fluorescein in water (10 add phosphate buffer pH 7.0, 1% DMSO) with a K_{A}=5×10⁶ M⁻¹. This complex is preferred by 3 kcal/mol over the binding of other fluorophores (Dansyl and pyrene) and N-Ac-Trp and 7 kcal/mol over N-Ac-Gly (Graph 1A). [2]Rotaxanes 2 and 3 are also selective for fluorescein. They bind fluorescein in water with a K_{A}=7×10 M⁻¹ and in DMSO with a K_{A}=9×10⁵ M⁻¹ (Graph 1B). This complex in both solvents is preferred by 1 kcal/mol over the binding of other fluorophores (Dansyl and pyrene) and N-Ac-Trp. Most likely, 4 and a 6 kcal/mol preferences exist for the association of fluorescein by rotaxanes 2 and 3 compared to Ac-Gly in DMSO and water, respectively. The values for Ac-Gly are taken from the studies of host-[2]rotaxane 1, which arise through a salt bridge between the Arg moiety of the ring and the carboxylate of Ac-Gly. This type of salt bridge should also exist and be the main driving force for the complex of rotaxane 2 and rotaxane 3 with Ac-Gly.

These results demonstrate that host-rotaxanes can be designed to selectively bind a guest. This selectivity appears to hold in a variety of solvents (DMSO and water) and cellular environments; transporter 3 delivers fluoresceinated guests throughout eukaryotic cells. Intracellular fluorescence could not be observed with rotaxane 1, which may be caused by its large binding affinity for fluorescein (K_{A}=5×10⁶ M⁻¹). If dissociation does not occur in the cell, the fluorescence of fluorescein would be quenched. The transport efficiency of rotaxane 1 will be investigated using the apoptosis assay and the inhibition of CaMK assay.

### Example 3

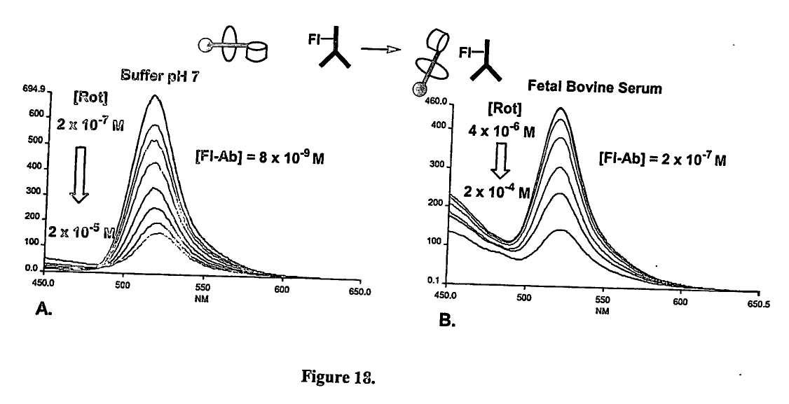

Rotaxane 3 associates with FITC-anti-goat (rabbit) antibody in buffer (K_{A}=8×10⁵ M⁻¹, phosphate, pH 7, FIG. 18A) and in full fetal bovine serum (K_{A}=1×10⁴ M⁻¹, FIG. 18B). The ADCT method relies on Fl-antibody association and cellular transport.

The following examples demonstrate transportation and transporter stability.

### Example 4

Cell-Transport Transporters 2 and 3 efficiently deliver impermeable fluorescein (Fl) into eukaryotic COS-7 cells in several minutes (data not shown). These experiments were performed in both phosphate buffer pH 7.0 and in serum medium (10% fetal bovine, results not shown). Transport in serum is significant because it demonstrates that the recognition of fluorescein occurs in a solution containing a variety of other possible guests (various amino acid side chains). Transporter 3 (transporter 2 was not tested) delivers fluorescein-tagged oligopeptides (Fl-KKALRAQEAVDAL and Fl-KAASLWVGPR) (data not shown). Fl-peptide transport is a very significant accomplishment since the oligopeptides have carboxylates, which impede membrane passage.

Host-[2]rotaxane 3 transports fluorescein at a lower concentration than cleft-[2]rotaxane 2 (6 μM versus 60 μM, both measured after 30 min). Host-[2]rotaxane 3 transports fluorescein (and other guests vide infra) to the nucleus to a greater extent than the cytoplasm, whereas cleft-[2]rotaxane 2 more uniformly transports fluorescein throughout the cell. Host-[2]rotaxane 3 is not toxic to the cells after a 12 h exposure time, whereas cleft-[2]rotaxane 2 caused cell blebbing after 12 h.

[2]Rotaxane 3 transports fluorescein-tagged oligopeptides (Fl-KKALRAQEAVDAL and Fl-KAASLWVGPR) and fluorescein-PKC inhibitor conjugate 3 into COS-7 cells at submicromolar concentrations. Fluorescein (only tested) has been transported by rotaxane 2 into ovarian cancer cell lines (NIH-OVCAR3 and ES-2). Peptide transport is a very significant accomplishment since generally dipeptides and tripeptides and highly cationic peptides transverse unaided through membranes. The oligopeptides have carboxylates, which impede membrane passage. PKC-conjugate is membrane permeable at a pH≦6.5 and used to locate intracellular protein kinase C. It is not permeable at pH values greater than 7.0. As evident by the results of the fluorescence microscopy experiments (data not shown), [2]rotaxane 3 dramatically enhances the permeability of conjugate 3 at pH 7.5. The amount transported at pH 7.5 appears to be even greater than the amount of conjugate 3 found within cells when it is exposed to cells at pH 6.0. Prolong exposure (14 hours) killed the cells, which is consistent with PKC inhibition.

A model transporter, which contains the key components and fluorescein, is highly cell-permeable (data not shown). Having a linked fluorescein, with its negative charges, should only reduce from cell-permeability of the model compound. This result suggests that transporters 2 and 3 are cell permeable without a guest and they will travel back and forth across membranes and cells to bring multiple drugs throughout a tumor. Furthermore, derivatizing the transporter, e.g. with half of a linker, should not hamper transport.

### Example 5

Selectivity. Transporters 2 and 3 were successfully designed to selectively bind fluorescein. They bind fluorescein in water with a K_{A}=7×10⁴ M⁻¹ and in DMSO with a K_{A}=9×10⁵ M⁻¹. This complex in both solvents is preferred by 1 kcal/mol over the binding of other fluorophores (Dansyl and pyrene) and N-Ac-Trp. More importantly, this selectivity appears to hold in cellular environments; transporter 3 delivers fluoresceinated guests throughout eukaryotic cells in the DMEM media, containing 10% fetal bovine serum. Transporters 2 and 3 also associate with fluoresceinated-anti-goat (rabbit) antibody in buffer (K_{A}=8×10⁵ M⁻¹, phosphate, pH 7) and in full fetal bovine serum (K_{A}=1×10⁴ M⁻¹).

### Example 6

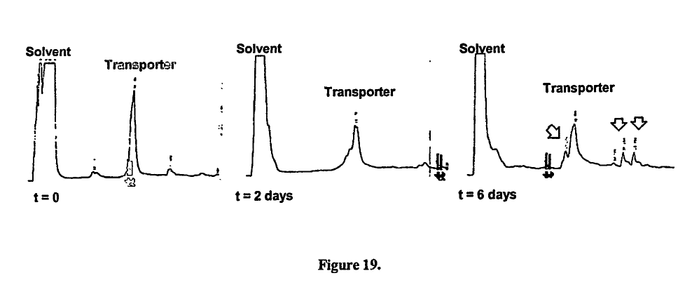

Serum Stability. Rotaxanes 2 and 3 are stable to serum medium. Each rotaxane was exposed to fetal calf bovine serum (5% DMSO/95% serum) for up to 6 days. HPLC analysis showed that, under these conditions, the rotaxanes are stable (rotaxane 2 results are shown in FIG. 19). These results are especially important for the ADCT method. Association with an antibody should protect the rotaxane from normal metabolic degradation. Once inside the tumor, the rotaxanes may be isolated from the normal metabolic pathways. A similar effect may be observed for rotaxanes with covalently linked targeting agents.

### Example 7

Methods for Matrigel Assays. Knowledge about tumors has been greatly facilitated by growing and investigating tumors in Matrigel. These three dimensional tumors are ideal for determining the ability of the transporters to penetrate tumors and for developing the ADCT method. A set of ovarian cancerous tumors will be grown in Matrigel. In one experiment, a transporter and fluorescein will be injected into the buffered solution (phosphate buffer pH 7.5) that surrounds the tumor (FIG. 17). After set time periods (1-7 days), a tumor will be dissected by slicing it, and the amount of fluorescence at various depths will be analyzed via fluorescence microscopy. A similar experiment was performed by Schalken who stained and dissected Matrigel to help determine the location of receptor c-MET in prostate epithelium To quantify the amount of fluorescein in the tissue, cores will be removed at various sites, fluorescein will be recovered by extraction, and the intensity of fluorescence of this solution will be measured.

### Example 8

A second experiment involves injecting a transporter directly into a tumor. Fluorescein will then be added to the buffer. The amount of fluorescein delivered into the tumor will be measured. If fluorescein (or a fluorescein-drug conjugate) is successfully delivered, this would suggest a more simplified anticancer therapy. In this therapy, transporters are injected directly into a tumor and a fluoresceinated drug or prodrug is administered (oral, intravenous, etc.). Additional transporters can be finely tuned for tumor attraction and deep tumor penetration. They are made from exchangeable parts and readily assembled.

### Example 9

Testing the ADCT Method with Matrigel. Once a suitable linker has been developed and the transporters tested, the ADCT method will be tested. The transporter will be attached to one end of a linker and the antibody will be attached to the other end.

Methods for detecting conjugate formation. For the sensitizer method, the amount of transporter linked to the antibody can be directly determined by performing the uv/vis and fluorescence assays described previously. For acid cleavable linkers, an indirect method will be performed to determine the amount of transporter attached. We have shown that transporters quench fluorescein upon association. Therefore, known amounts of fluorescein will be added to an aliquot of a newly formed antibody-transporter conjugate. The degree of fluorescence quenching will indicate the concentration of the transporter. Knowing the initial concentration of antibody, we can determine the number of transporters linked. These calculations assume that the association constant for transporter-fluorescein complex formation is the equivalent to the ones already measured (preliminary results).

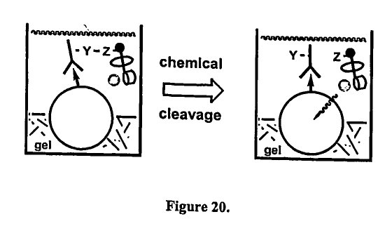

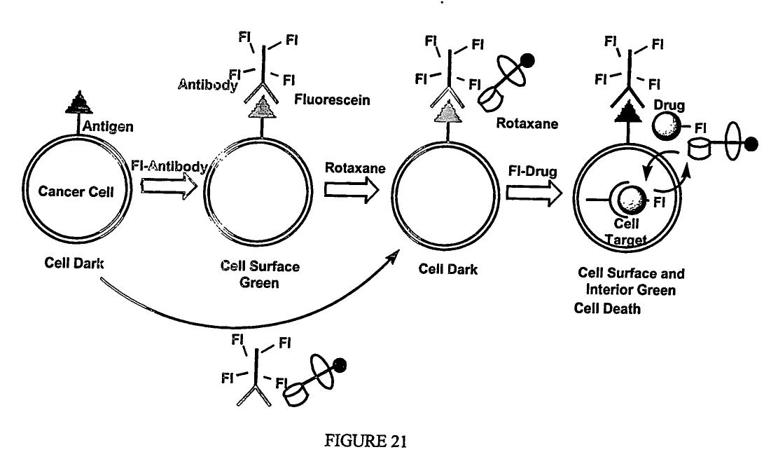

Methods for Matrigel Assays. Antibody-transporter conjugates will be added to a buffered solution in a well containing a tumor in Matrigel (FIGS. 20 and 21). ELISA performed on the tumor will show the ability of the antibody to bind the tumor. Acid sensitive linkers will be cleaved by switching the buffer that surrounds this tumor or a tumor without the ELISA components (in case these components interfere with the ADCT method) from pH 7.5 to pH 6.0 to simulate the acidic environment found around tumors. For light activated linkers, long wavelength light will be used to cleave the transporter from the antibody. For both methods, fluorescein will be added after transporter release, and the amount of fluorescein delivered at various times will be determined using the methods described above. In separate experiments, fluorescein will be added at set time periods (1, 2, 3 days etc.) after the linker is cleaved. These experiments will indicate the flexibility of the ADCT method in terms of when a Fl-drug can be administered.

Fluoresceinated drug conjugates, e.g., fluoresceinated nitracine, may also be used in these Matrigel assays. Nitracrine, originally developed as a traditional anticancer drug, is a potent hypoxic cell radiosensitizer, and hypoxia-selective cytotoxin in cell culture. Nitracine has a acridine ring system. A combination of the ADCT method and cancer cell selective prodrugs may result in a highly selective anticancer therapy.

1. A pharmaceutical composition comprising a host-rotaxane and a guest molecule in a pharmaceutically-acceptable carrier, wherein the guest molecule comprises an active agent.

2. The composition according to claim 1, wherein the host-rotaxane comprises at least one linear component disposed in at least one cyclic component.

3. The composition according to claim 1, wherein the host-rotaxane comprises at least one blocking group.

4. The composition according to claim 2, wherein the at least one of the blocking group comprises a linker for associating the guest molecule to the host-rotaxane.

5. The composition according to claim 4, wherein the linker comprises a cyclic aliphatic ethers, non-cyclic aliphatic ethers, cyclic aromatic compounds, non-cyclic aromatic compounds, anionic species, cationic species, and functionalized constructions thereof.

6. The composition according to claim 1, wherein the guest molecule comprises proteins, peptides, amino acids, aromatic compounds, inorganic cations, inorganic anions, organic cations, organic anions, sugars, DNA, RNA, nucleotides, phosphates, phospholipids, fatty acids, steroids, isoprene derivatives.

7. The composition according to claim 2, wherein the host-rotaxane further comprises at least one recognition element.

8. The composition according to claim 7, wherein the at least one recognition element is attached to at least one cyclic component.

9. The composition according to claim 3, wherein the at least one recognition element is capable of forming an interaction with the linker, the guest molecule or a combination thereof.

10. The composition according to claim 9, wherein the interaction comprises hydrogen bonds, electrostatic interactions, dispersion interactions or a combination thereof.

11. The composition according to claim 7, wherein the at least one recognition element comprises carboxylates, ammonium ions, guanidinium ions, imidazolium ions, phosphates, aromatic rings, aliphatic groups, alcohols, amides, carboxylates, sulfhydryls, or combinations thereof.

12. The composition according to claim 4, wherein the host-rotaxane comprises at least one polar recognition element on at least one cyclic component.

13. The composition according to claim 1, wherein the agent is a therapeutic agent.

14. The composition according to claim 1, wherein the composition further comprises a binding element.

15. The composition according to claim 14, wherein the binding element comprises a marking element.

16. The composition according to claim 15, wherein the marking element is a fluorophore.

17. A composition comprising a host-rotaxane and an agent.

18. The composition according to claim 17, wherein the agent comprises a vaccine, a drug, a prodrug, or a derivative or an analog thereof.

19. A method of delivering an agent to a subject, comprising administering to the subject a composition comprising a host-rotaxane and an agent.

20. The method according to claim 19, wherein the agent is administered prior to, concurrently, or subsequently to the administration of the host-rotaxane.

21. The method according to claim 19, wherein the agent comprises a vaccine, a drug, a prodrug, or a derivative or an analog thereof.

22. The method according to claim 19, wherein the agent is administered to target cancers, tumors, malignancies, uncontrolled tissue, cellular proliferation, or a combination thereof.

23. The method according to claim 19, wherein the composition is administered orally, parenterally, intrasystemically, intraperitoneally, topically or combinations thereof.

24. The method according to claim 19, wherein the composition comprises a pharmaceutically acceptable carrier.

25. The method according to claim 14, wherein the carrier comprises a solid, semisolid, liquid filler, diluent, or encapsulating material.

26. The method according to claim 19, further comprising a subsequent administration of an agent to the individual.

27. The method according to claim 20, further comprising a subsequent administration to the individual of a guest molecule bound with an agent.

28. A method of treating cancerous cells in an individual, comprising administering to the individual a composition comprising a host-rotaxane, a guest molecule and an agent bound to the host-rotaxane, wherein the composition delivers the agent to the cancerous cells.

29. The method according to claim 28, wherein the cancerous cells are a tumor.

30. The method according to claim 28, wherein the agent is a drug, a prodrug, or a derivative or an analog thereof.

31. A method for diagnosing cancerous cells in an individual, comprising administering to the individual a composition comprising a host-rotaxane, a guest molecule and a marking element and diagnosing the cancerous cells in the individual.

32. The method according to claim 31, wherein the marking element comprises a fluorophore.

33. The method according to claim 31, wherein the cancerous cells are diagnosed by imaging.

34. The method according to claim 19, wherein the host-rotaxane is conjugated to a target-binding moiety.
